(19) United States
(12) Patent Application Publication
(10) Pub. No.: US 2012/0315267 A1
(43) Pub. Date: Dec. 13, 2012

(54) NOVEL USES

(75) Inventors: Stephanie Jane Clegg, Stevenage (GB); Eric Dobrzynski, King of Prussia, PA (US); Jonathan Henry Ellis, Stevenage (GB); Volker Germaschewski, Stevenage (GB); Alexis Paul Godillot, King of Prussia, PA (US); Zdenka Ludmila Jonak, King of Prussia, PA (US); Alan Peter Lewis, Stevenage (GB); John Richard White, King of Prussia, PA (US)

(73) Assignee: GLAXOSMITHKLINE LLC, PHILADELPHIA, PA (US)

(21) Appl. No.: 13/577,923

(22) PCT Filed: Feb. 9, 2011
(86) PCT No.: PCT/US11/24123

§ 371 Date: Aug. 9, 2012

Related U.S. Application Data
(60) Provisional application No. 61/302,636, filed on Feb. 9, 2010.

(51) Int. Cl. A61K 39/395; C12N 15/63; A61P 19/02; A61P 11/06; A61P 9/10; A61P 1/00; A61P 1/04; A61P 37/06; A61P 35/00; A61P 11/00; C12N 5/10; C12N 1/19; C12N 1/21; C12N 1/15; C07K 16/00

(52) U.S. Cl. 424/130.1; 530/387.1; 435/320.1; 435/326; 435/254.2; 435/252.33; 435/252.31; 435/252.34; 435/252.35; 435/254.21; 435/254.23; 435/254.22; 435/254.3; 435/254.6; 435/254.5; 435/254.11; 435/252.3

(57) ABSTRACT

The present invention relates to antibodies which multiple antigen specificities and their use in treating human diseases

### FIELD OF THE INVENTION

The present invention relates to an antibody which has multiple specificities. In particular the antibody of the present invention has the ability to bind to (i.e. cross react with) up to four antigens selected from the group consisting of human IL-8, Gro-alpha, Gro-beta, Gro-gamma, GCP2, NAP2, and ENA-78. The present invention also concerns with methods of treating diseases or disorders characterised by elevated or unbalanced level of one or more of human IL-8, Gro-alpha, Gro-beta, Gro-gamma, GCP2, NAP2, and ENA-78 particularly COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis with said antibody.

### BACKGROUND OF THE INVENTION

Published data and reports indicate that the members of the ELRCXC subfamily of CXCL chemokines are elevated in a number of diseases. There are a total of 16 CXCL family members. The chemokines are reported to be up-regulated in a number of inflammatory diseases, including COPD, in which CXCL1-3, 5, and 8, also known as Gro- - , - , - (Haskill, S., et al. Proc. Natl. Acad. Sci., 1990: 87, 7732-7736), ENA-78 (Wang, D. and Richmond, A., Cytokine Reference. Oppenheim, J. J. and Feldman, M. ed., Academic Press, London, 1023-1027, Powerm C. A. et al. Gene., 1994: 151, 333-334), and IL-8 (Iizasa, H. and Matsushima, K., Cytokine Reference. Oppenheim, J. J. and Feldman, M. ed., Academic Press, London, 1061-1067, Matsushima, K. et al., J. Exp. Med. 1988: 167, 1883-1893) respectively (Am. J. Respir. Crit. Care Med., 163: 349-355, 2001, Am. J. Respir. Crit. Care Med., 168: 968-975, 2003, Thorax, 57: 590-595, 2002). It has be postulated that prolonged and elevated expression of these chemokines could be involved in the development of diseases such as COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, or endometriosis. These CXC chemokines are known to stimulate neutrophil chemotaxis by engaging and activating the CXCR1 and/or CXCR2 receptors. Thus the inhibition of these chemokines could prevent inflammatory cells from infiltrating the lung tissue and thus prevent tissue damage. The present invention is directed to inhibiting the activation of CXCR1 and CXCR2 receptors by using an antibody having the ability to bind to (i.e. cross react with) up to four antigens selected from the group consisting of human IL-8, Gro-alpha, Gro-beta, Gro-gamma, GCP2, NAP2, and ENA-78.

### SUMMARY OF THE INVENTION

The present invention relates to an antibody (immunoglobulin) which has multiple specificities contained within one immunoglobulin. In particular the antibody of the present invention has the ability to bind to (i.e. cross react with) up to four antigens selected from the group consisting of human IL-8, Gro-alpha, Gro-beta, Gro-gamma, GCP2, NAP2, and ENA-78. The present invention also concerns with methods of treating diseases or disorders characterised by elevated or unbalanced level of one or more of human IL-8, Gro-alpha, Gro-beta, Gro-gamma, GCP2, NAP2, and ENA-78, particularly COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis with said antibody.

In one aspect, the present invention relates to an isolated antibody which has the ability to bind to (i.e. cross react with) up to four antigens selected from the group consisting of human IL-8, Gro-alpha, Gro-beta, Gro-gamma, GCP2, NAP2, and ENA-78 The definition of antibody includes an antigen binding portion (or fragment) of the antibody such that the antigen binding portion (or fragment) binds (i.e. cross react with) up to four antigens selected from the group consisting of human IL-8, Gro-alpha, Gro-beta, Gro-gamma, GCP2, NAP2, and ENA-78. The antibody of the invention is preferably murine monoclonal, chimeric, human or humanized.

In one embodiment the present invention comprises a method of decreasing the neutrophil chemotaxis through inhibition of CXCR1 and CXCR2 receptor activation by neutralizing up to four antigens selected from the group consisting of human IL-8, Gro-alpha, Gro-beta, Gro-gamma, GCP2, NAP2, and ENA-78 with an antibody of the present invention.

In one embodiment the present invention relates to a method of decreasing the neutrophil chemotaxis in a patient in need thereof by administering an antibody of the present invention.

In one embodiment, the antibody of the present invention can be generated by a method comprising the steps of using RIMMs (Kilpatrick, K. E., et al. Hybridoma. 1997: 16, 381.) type protocol using a mixture (cocktail) of human IL-8, Gro-alpha, Gro-beta, Gro-gamma, and ENA-78 together with a set of five multiple antigenic peptides (MAPs) each MAP unit having one separate sequence from polypeptides of ID NOs: 89-93.

SEQ ID NO: 89
LATELRSQSLQTLQG
SEQ ID NO: 90
SAKELRSQSIKTYSK
SEQ ID NO: 91
LRELRSVSLQTTQG
SEQ ID NO: 92
SPGPHSAQTEVIAT
SEQ ID NO: 93
ESGPHSANTEIIVK

Without being bound by theory, MAPs serve two functions within the immunization protocol. First, MAPs allow for a selective multiple presentation of a known target amino acid sequence to the host immune system. Secondly, the increase in mass, due to multiple copies of the sequence linked via a core, such as, but not limited to lysine, increases the immunogenicity of the sequence over that of individual peptides (Francis, J. P., et al., Immunology, 1991: 73; 249, Schott, M. E., et al., Cell. Immuno. 1996: 174: 199-209, Tam, J. P. Proc. Natl. Acad. Sci. 1988: 85; 5409-5413).

The MAPs used to generate this invention are comprised of multiple copies of the conserved target sequences (e.g. SEQ ID NOs: 89-93) found with and around the ELRCXC and GPHCA regions of target chemokines Exemplary MAP set is depicted in FIG. 1.

In one embodiment, the antibody of the present invention can be generated by a method comprising the steps of:
a. injecting into a mouse a mixture of human IL-8, Gro-alpha, Gro-beta, Gro-gamma, and ENA-78 in complete Freund's adjuvant (cFA);
b. injecting into the mouse a mixture of human IL-8, Gro-alpha, Gro-beta, Gro-gamma, and ENA-78 in incomplete Freund's adjuvant (iFA); and
c. injecting into the mouse a mixture of human IL-8, Gro-alpha, Gro-beta, Gro-gamma, and ENA-78, and a set of five multiple antigenic peptides (MAPs), each MAP unit having one separate sequence from polypeptides of ID NOs: 89-93 in incomplete Freund's adjuvant;
d. isolating B cells from the mouse;
e. fusing the B cells with myeloma cells to form immortal hybridoma cells that secrete the desired antibody; and
f. isolating the antibody from the culture supernatant of the hybridoma.
If desired, one can optionally inject into the mouse a mixture of human IL-8, Gro-alpha, Gro-beta, Gro-gamma, and ENA-78, and a set of MAPs comprising amino acid sequences of SEQ ID NOs: 89-93 in PBS between steps c and d.

In another embodiment, an antibody of the present invention is generated by a method comprising the steps of:
a. injecting into a mouse a set of five multiple antigenic peptides (MAPs) each MAP unit having one separate sequence from polypeptides of ID NOs: 89-93 (hereinafter also referred to as the MAP set) in complete Freund's adjuvant;
b. injecting into the mouse the MAP set in incomplete Freund adjuvant;
c. injecting into the mouse a mixture of all human IL-8, Gro-alpha, Gro-beta, Gro-gamma, and ENA-78, and the MAP set in incomplete Freund's adjuvant;
d. isolating B cells from the mouse; and
e. fusing the B cells with myeloma cells to form immortal hybridoma cells that secrete the desired antibody; and
f. isolating the antibody from the culture supernatant of the hybridoma.
If desired, one can optionally inject into the mouse a mixture of human IL-8, Gro-alpha, Gro-beta, Gro-gamma, and ENA-78, and a set of MAPs having SEQ ID NOs: 89-93 in PBS between steps c and d.

In another embodiment, an antibody of the present invention is generated by a method comprising the steps of:
a. injecting into a mouse a cocktail of human recombinant purified chemokines (human IL-8, Gro-alpha, Gro-beta, Gro-gamma, and ENA-78) in complete Freund's adjuvant;
b. injecting into the mouse the chemokine cocktail in incomplete Freund's adjuvant;
c. isolating B cells from the mouse; and
d. fusing the B cells with myeloma cells to form immortal hybridoma cells that secrete the desired antibody; and
e. isolating the antibody from the culture supernatant of the hybridoma.
   In another embodiment, the present invention concerns an antibody made by the foregoing processes.

In one embodiment, an antibody comprises at least one variable region selected from (i) the amino acid SEQ ID NO: 1, 2, 3, 4, 5, 6, 7, 8, 9, 10, 11, 12, 13, 14, 15, 16, 17, 18, 19, 20, 21, or 22; or (ii) an amino acid sequence which is at least 90%, 95%, 98% or 99% identical to any one of the amino acid sequences of (i) above.

In one embodiment, the present invention concerns a hybridoma which produces a monoclonal antibody comprising heavy and light chain variable region comprising the amino acid sequences of SEQ ID NO:1, 3, 5, 7, 9, 11, 13, 15, 17, 19, or 21, and SEQ ID NO: 2, 4, 6, 10, 12, 14, 16, 18, 20, or 22, respectively, and conservative sequence modifications thereof.

In one embodiment, the present invention concerns a hybridoma which produces a monoclonal antibody comprising heavy or light chain variable region comprising the amino acid sequences of SEQ ID NO:1, 3, 5, 7, 9, 11, 13, 15, 17, 19, or 21, or SEQ ID NO: 2, 4, 6, 10, 12, 14, 16, 18, 20, or 22, respectively, and conservative sequence modifications thereof.

In one embodiment, the present invention concerns an antibody comprising heavy or light chain variable region comprising the amino acid sequences of SEQ ID NO:1, 3, 5, 7, 9, 11, 13, 15, 17, 19, or 21; or SEQ ID NO: 2, 4, 6, 10, 12, 14, 16, 18, 20, or 22, respectively, and conservative sequence modifications thereof.

In one embodiment, the present invention concerns an antibody comprising heavy and light chain variable region comprising the amino acid sequences of SEQ ID NO:1, 3, 5, 7, 9, 11, 13, 15, 17, 19, or 21, and SEQ ID NO: 2, 4, 6, 10, 12, 14, 16, 18, 20, or 22, respectively, and conservative sequence modifications thereof.

In one embodiment, an antibody comprises heavy and light chain variable regions comprising the amino acid sequences of SEQ ID NO:1 and SEQ ID NO:2, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:1 and SEQ ID NO:2, respectively.

In one embodiment, an antibody comprises CDR sequences of SEQ ID NOs: 23, 24, 25, 26, 27, and 28; or one or more of the CDR sequences can be conservative sequence modifications of the sequences SEQ ID NOs: 23, 24, 25, 26, 27, and 28.

In one embodiment, the present invention relates to an hybridoma which produces an antibody which comprises CDR sequences of SEQ ID NOs: 23, 24, 25, 26, 27, and 28.

In one embodiment, the present invention relates to a recombinant eukaryotic or prokaryotic cell which produces an antibody which comprises CDR sequences of SEQ ID NOs: 23, 24, 25, 26, 27, and 28.

In one embodiment, an antibody comprises at least one CDR sequence selected from (i) SEQ ID NO: 23, 24, 25, 26, 27, or 28; or (ii) a conservative sequence modification of the sequences listed in (i).

In one embodiment, an antibody comprises a polypeptide of SEQ ID NO:25.

In one embodiment, an antibody comprises at least four CDR sequences selected from the group consisting of SEQ ID NOs: 23, 24, 25, 26, 27, and 28; or one or more of the CDR sequences can be conservative sequence modifications of the sequences listed in SEQ ID NOs: 23, 24, 25, 26, 27, and 28.

In one embodiment, an antibody comprises heavy and light chain variable regions which comprise the CDR amino acid sequences of SEQ ID NOs: 23, 24, and 25, and SEQ ID NOs: 26, 27, and 28, respectively, or one or more of the CDR sequences can be conservative sequence modifications of the sequences listed in SEQ ID NOs: 23, 24, 25, 26, 27, and 28.

In one embodiment, an antibody of the present invention comprises:
i) CDRH1 as set out in SEQ ID NO. 23 or a variant of SEQ ID NO. 23 wherein Tyr32 is substituted for Ile, His, Phe, Thr, Asn, Cys, Glu or Asp and/or Gly33 is substituted for Tyr, Ala, Trp, Thr, Leu or Val and/or Met34 is substituted for Ile, Val or Trp and/or Ser35 is substituted for His, Glu, Asn, Gln, Tyr or Thr;
ii) CDRH2 as set out in SEQ ID NO. 24 or a variant of SEQ ID NO. 24 wherein Trp50 is substituted for Arg, Glu, Tyr, Gly, Gln, Val, Leu, Asn, Lys or Ala and/or Ile51 is substituted for Leu, Val, Thr, Ser or Asn and/or Asn52 is substituted for Asp, Leu, Ser or Tyr and/or Tyr53 is substituted for Ala, Gly, Ser, Lys, Thr or Asn and/or Ser54 is substituted for Asn, Thr, Lys, Asp or Gly and/or Val56 is substituted for Tyr, Arg, Glu, Asp, Gly, Ser or Ala and/or Thr58 is substituted for Lys, Asn, Ser, Asp, Arg, Gly, Phe or Tyr;
iii) CDRH3 as set out in SEQ ID NO. 25 or a variant of SEQ ID NO. 25 wherein Val102 is substituted for Tyr, His, Ile, Ser, Asp or Gly;
iv) CDRL1 as set out in SEQ ID NO. 26 or a variant of SEQ ID NO. 26 wherein Asn28 is substituted for Ser, Asp, Thr or Glu and/or Ile29 is substituted for Val and/or Tyr30 is substituted for Asp, Leu, Val, Ile, Ser, Asn, Phe, His, Gly or Thr and/or Ser31 is substituted for Asn, Thr, Lys or Gly and/or Asn32 is substituted for Phe, Tyr, Ala, His, Ser or Arg and/or Leu33 is substituted for Met, Val, Ile or Phe and/or Ala34 is substituted for Gly, Asn, Ser, His, Val or Phe;
v) CDRL2 as set out in SEQ ID NO. 27 or a variant of SEQ ID NO. 27 wherein Ala51 is substituted for Thr, Gly or Val; and
vi) CDRL3 as set out in SEQ ID NO. 28 or a variant of SEQ ID NO. 28 wherein Gln89 is substituted for Ser, Gly, Phe or Leu and/or His90 is substituted for Gln or Asn, Phe91 is substituted for Asn, Gly, Ser, Arg, Asp, His, Thr, Tyr or Val and/or Trp92 is substituted for Asn, Tyr, Thr, Ser, Arg, Gln, His, Ala or Asp and/or Thr93 is substituted for Glu, Asn, Gly, His, Ser, Arg or Ala and/or Thr94 is substituted for Asp, Tyr, Val, Leu, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 23;

ii) CDRH2 as set out in SEQ ID NO. 24;

iii) CDRH3 as set out in SEQ ID NO. 25;

iv) CDRL1 as set out in SEQ ID NO. 26;

v) CDRL2 as set out in SEQ ID NO. 27;

vi) CDRL3 as set out in SEQ ID NO. 28;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe.

In one embodiment, an antibody of the present invention comprises:

i) CDRH3 as set out in SEQ ID NO. 25

ii) CDRH1 as set out in SEQ ID NO. 23

iii) CDRH2 as set out in SEQ ID NO. 24

iv) CDRL1 as set out in SEQ ID NO. 26

v) CDRL2 as set out in SEQ ID NO. 27

vi) CDRL3 as set out in SEQ ID NO. 28;

vii) the heavy chain framework comprising the following residues:
Position 2 Ile
Position 4 Leu
Position 20 Ile
Position 22 Cys
Position 24 Ala
Position 26 Gly
Position 29 Phe
Position 36 Trp
Position 47 Trp
Position 48 Met
Position 69 Phe
Position 71 Leu
Position 78 Ala
Position 80 Leu
Position 90 Tyr
Position 92 Cys
Position 94 Arg; and

viii) the light chain framework comprising the following residues:
Position 2 Ile
Position 3 Gln
Position 4 Met
Position 23 Cys
Position 35 Trp
Position 36 Tyr
Position 46 Leu
Position 49 Tyr
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 23, 24, and 25; or SEQ ID NOs: 23, 24, and 25, respectively.

In one embodiment, the present invention relates to an expression vector comprising a nucleotide sequence encoding a CDR sequence of an antibody selected from SEQ ID NO: 23, 24, 25, 26, 27, or 28.

In one embodiment, the present invention relates to an expression vector comprising nucleotide sequences encoding at least four CDR sequences of an antibody selected from the group consisting of SEQ ID NOs: 23, 24, 25, 26, 27, and 28.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 23, 24, and 25; and a second DNA sequence encoding at least the variable domain of the immunoglobulin light chain comprising CDR domains of SEQ ID NOs: 26, 27, and 28; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.

In one embodiment, an antibody comprises heavy and light chain variable regions comprising the amino acid sequences of SEQ ID NO:3 and SEQ ID NO:4, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO: 3 and SEQ ID NO:4, respectively.

In one embodiment, an antibody comprises CDR sequences of SEQ ID NOs: 29, 30, 31, 32, 33, and 34; or one or more of the CDR sequences can be conservative sequence modifications of the sequences SEQ ID NOs: 29, 30, 31, 32, 33, and 34.

In one embodiment, the present invention relates to an hybridoma which produces an antibody which comprises CDR sequences of SEQ ID NOs: 29, 30, 31, 32, 33, and 34. In one embodiment, the present invention relates to a recombinant eukaryotic or prokaryotic cell which produces an antibody which comprises CDR sequences of SEQ ID NOs: 29, 30, 31, 32, 33, and 34.

In one embodiment, an antibody comprises at least one CDR sequence selected from (i) SEQ ID NO: 29, 30, 31, 32, 33, or 34; or (ii) a conservative sequence modification of the sequences listed in (i).

In one embodiment, an antibody comprises a polypeptide of SEQ ID NO:31.

In one embodiment, an antibody comprises at least four CDR sequences selected from the group consisting of SEQ ID NOs: 29, 30, 31, 32, 33, and 34; or one or more of the CDR sequences can be conservative sequence modifications of the sequences listed in SEQ ID NOs: 29, 30, 31, 32, 33, and 34.

In one embodiment, an antibody comprises heavy and light chain variable regions which comprise the CDR amino acid sequences of SEQ ID NOs: 29, 30, and 31, and SEQ ID NOs: 32, 33, and 34, respectively; or one or more of the CDR sequences can be conservative sequence modifications of the sequences listed in SEQ ID NOs: 29, 30, 31, 32, 33, and 34.

In one embodiment, an antibody of the present invention comprises:
i) CDRH1 as set out in SEQ ID NO. 29 or a variant of SEQ ID NO. 29 wherein Tyr32 is substituted for Ile, His, Phe, Thr, Asn, Cys, Glu or Asp and/or Tyr33 is substituted for Gly, Ala, Trp, Thr, Leu or Val and/or Met34 is substituted for Ile, Val or Trp and/or Asn35 is substituted for His, Glu, Ser, Gln, Tyr or Thr;
ii) CDRH2 as set out in SEQ ID NO. 30 or a variant of SEQ ID NO. 30 wherein Asp50 is substituted for Arg, Glu, Trp, Tyr, Gly, Gln, Val, Leu, Asn, Lys or Ala and/or Val51 is substituted for Leu, Ile, Thr, Ser or Asn and/or Asn52 is substituted for Asp, Leu, Ser or Tyr and/or Asp53 is substituted for Ala, Gly, Tyr, Ser, Lys, Thr or Asn and/or Asp54 is substituted for Asn, Thr, Lys, Ser or Gly and/or Asp56 is substituted for Tyr, Arg, Glu, Val, Gly, Ser or Ala and/or Thr58 is substituted for Lys, Asn, Ser, Asp, Arg, Gly, Phe or Tyr;
iii) CDRH3 as set out in SEQ ID NO. 31 or a variant of SEQ ID NO. 32 wherein Val102 is substituted for Tyr, His, Ile, Ser, Asp or Gly;
iv) CDRL1 as set out in SEQ ID NO. 32 or a variant of SEQ ID NO. 32 wherein Asp28 is substituted for Ser, Asn, Thr or Glu and/or Ile29 is substituted for Val and/or Arg30 is substituted for Tyr, Asp, Leu, Val, Ile, Ser, Asn, Phe, His, Gly or Thr and/or Asn31 is substituted for Ser, Thr, Lys or Gly and/or Tyr32 is substituted for Phe, Asn, Ala, His, Ser or Arg and/or Leu33 is substituted for Met, Val, Ile or Phe and/or Asn34 is substituted for Ala, Asn, Ser, His, Val or Phe;
v) CDRL2 as set out in SEQ ID NO. 33 or a variant of SEQ ID NO. 33 wherein Thr51 is substituted for Ala, Gly or Val; and
vi) CDRL3 as set out in SEQ ID NO. 34 or a variant of SEQ ID NO. 34 wherein Gln89 is substituted for Ser, Gly, Phe or Leu and/or Gln90 is substituted for His or Asn, Ala91 is substituted for Asn, Phe, Gly, Ser, Arg, Asp, His, Thr, Tyr or Val and/or Asn92 is substituted for Trp, Tyr, Thr, Ser, Arg, Gln, His, Ala or Asp and/or Thr93 is substituted for Glu, Asn, Gly, His, Ser, Arg or Ala and/or Leu94 is substituted for Asp, Tyr, Val, Thr, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 29

ii) CDRH2 as set out in SEQ ID NO. 30

iii) CDRH3 as set out in SEQ ID NO. 31

iv) CDRL1 as set out in SEQ ID NO. 32

v) CDRL2 as set out in SEQ ID NO. 33

vi) CDRL3 as set out in SEQ ID NO. 34;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe

In one embodiment, an antibody of the present invention comprises:

i) CDRH3 as set out in SEQ ID NO. 31;

ii) CDRH1 as set out in SEQ ID NO. 29;

iii) CDRH2 as set out in SEQ ID NO. 30;

iv) CDRL1 as set out in SEQ ID NO. 32;

v) CDRL2 as set out in SEQ ID NO. 33;

vi) CDRL3 as set out in SEQ ID NO. 34;

vii) the heavy chain framework comprising the following residues:
Position 2 Val
Position 4 Leu
Position 20 Ile
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Tyr
Position 92 Cys
Position 94 Arg; and

viii) the light chain framework comprising the following residues:
Position 2 Ile
Position 3 Gln
Position 4 Met
Position 23 Cys
Position 35 Trp
Position 36 Phe
Position 46 Leu
Position 49 Tyr
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 29, 30, and 31; or SEQ ID NOs: 32, 33, and 34, respectively.

In one embodiment, the present invention relates to an expression vector comprising a nucleotide sequence encoding a CDR sequence of an antibody selected from SEQ ID NO: 29, 30, 31, 32, 33, or 34.

In one embodiment, the present invention relates to an expression vector comprising nucleotide sequences encoding at least four CDR sequences of an antibody selected from the group consisting of SEQ ID NOs: 29, 30, 31, 32, 33, and 34.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 29, 30, and 31; and a second DNA sequence encoding at least the variable domain of the immunoglobulin light chain comprising CDR domains of SEQ ID NOs: 32, 33, and 34; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.

In one embodiment, an antibody comprises heavy and light chain variable regions comprising the amino acid sequences of SEQ ID NO:5 and SEQ ID NO:6, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:5 and SEQ ID NO:6, respectively.

In one embodiment, an antibody comprises CDR sequences of SEQ ID NOs: 35, 36, 37, 38, 39, and 40; or one or more of the CDR sequences can be conservative sequence modifications of the sequences SEQ ID NOs: 35, 36, 37, 38, 39, and 40.

In one embodiment, the present invention relates to an hybridoma which produces an antibody which comprises CDR sequences of SEQ ID NOs: 35, 36, 37, 38, 39, and 40.

In one embodiment, the present invention relates to a recombinant eukaryotic or prokaryotic cell which produces an antibody which comprises CDR sequences of SEQ ID NOs: 35, 36, 37, 38, 39, and 40.

In one embodiment, an antibody comprises at least one CDR sequence selected from (i) SEQ ID NO: 35, 36, 37, 38, 39, or 40; or (ii) a conservative sequence modification of the sequences listed in (i).

In one embodiment, an antibody comprises a polypeptide of SEQ ID NO:37.

In one embodiment, an antibody comprises at least four CDR sequences selected from the group consisting of SEQ ID NOs: 35, 36, 37, 38, 39, and 40; or one or more of the CDR sequences can be conservative sequence modifications of the sequences listed in SEQ ID NOs: 35, 36, 37, 38, 39, and 40.

In one embodiment, an antibody comprises heavy and light chain variable regions which comprise the CDR amino acid sequences of SEQ ID NOs: 35, 36, and 37, and SEQ ID

NOs: 38, 39, and 40, respectively; or one or more of the CDR sequences can be conservative sequence modifications of the sequences listed in SEQ ID NOs: 35, 36, 37, 38, 39, and 40.

In one embodiment, an antibody of the present invention comprises:
i) CDRH1 as set out in SEQ ID NO. 35 or a variant of SEQ ID NO. 35 wherein Asn32 is substituted for Ile, His, Phe, Thr, Tyr, Cys, Glu or Asp and/or Asp33 is substituted for Tyr, Ala, Trp, Gly, Thr, Leu or Val and/or Ile34 is substituted for Met, Val or Trp and/or Asn35 is substituted for His, Glu, Ser, Gln, Tyr or Thr;
ii) CDRH2 as set out in SEQ ID NO. 36 or a variant of SEQ ID NO. 36 wherein Trp50 is substituted for Arg, Glu, Tyr, Gly, Gln, Val, Leu, Asn, Lys or Ala and/or Ile51 is substituted for Leu, Val, Thr, Ser or Asn and/or Phe52 is substituted for Asp, Leu, Asn, Ser or Tyr and/or Gly53 is substituted for Ala, Tyr, Ser, Lys, Thr or Asn and/or Asp54 is substituted for Asn, Thr, Lys, Ser or Gly and/or Ser56 is substituted for Tyr, Arg, Glu, Asp, Val, Ser or Ala and/or Lys58 is substituted for Thr, Asn, Ser, Asp, Arg, Gly, Phe or Tyr;
iii) CDRH3 as set out in SEQ ID NO. 37 or a variant of SEQ ID NO. 37 wherein Tyr102 is substituted for Val, His, Ile, Ser, Asp or Gly;
iv) CDRL1 as set out in SEQ ID NO. 38 or a variant of SEQ ID NO. 38 wherein Asp28 is substituted for Ser, Asn, Thr or Glu and/or Val29 is substituted for Ile and/or Gly30 is substituted for Asp, Leu, Val, Ile, Ser, Asn, Phe, His, Tyr or Thr and/or Thr31 is substituted for Asn, Ser, Lys or Gly and/or Ala32 is substituted for Phe, Tyr, Asn, His, Ser or Arg and/or Val33 is substituted for Met, Leu, Ile or Phe and/or Ala34 is substituted for Gly, Asn, Ser, His, Val or Phe;
v) CDRL2 as set out in SEQ ID NO. 39 or a variant of SEQ ID NO. 39 wherein Thr51 is substituted for Ala, Gly or Val; and
vi) CDRL3 as set out in SEQ ID NO. 40 or a variant of SEQ ID NO. 40 wherein His89 is substituted for Gln, Ser, Gly, Phe or Leu and/or Gln90 is substituted for His or Asn, Tyr91 is substituted for Asn, Gly, Ser, Arg, Asp, His, Thr, Phe or Val and/or Asn92 is substituted for Trp, Tyr, Thr, Ser, Arg, Gln, His, Ala or Asp and/or Asn93 is substituted for Glu, Thr, Gly, His, Ser, Arg or Ala and/or Tyr94 is substituted for Asp, Thr, Val, Leu, His, Asn, Ile, Trp, Pro or Ser and/or Leu96 is substituted for Pro, Trp, Tyr, Arg, Ile or Phe.

In one embodiment, an antibody of the present invention comprises:

i) CDRH1 as set out in SEQ ID NO. 35

ii) CDRH2 as set out in SEQ ID NO. 36

iii) CDRH3 as set out in SEQ ID NO. 37

iv) CDRL1 as set out in SEQ ID NO. 38

v) CDRL2 as set out in SEQ ID NO. 39

vi) CDRL3 as set out in SEQ ID NO. 40; and

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Thr, Val, Ala or Leu
Position 78 Ala, Leu, Val, Tyr or Phe
Position 80 Leu or Met,
Position 90 Tyr or Phe
Position 92 Cys; and
Position 94 Thr, Arg, Lys, Gly, Ser, His or Asn

viii) the light chain framework comprising the following residues:
Position 2 Ile, Leu or Val
Position 3 Val, Gln, Leu or Glu
Position 4 Met or Leu
Position 23 Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe.

In one embodiment, an antibody of the present invention comprises:

i) CDRH3 as set out in SEQ ID NO. 37

ii) CDRH1 as set out in SEQ ID NO. 35

iii) CDRH2 as set out in SEQ ID NO. 36

iv) CDRL1 as set out in SEQ ID NO. 38

v) CDRL2 as set out in SEQ ID NO. 39

vi) CDRL3 as set out in SEQ ID NO. 40;

vii) the heavy chain framework comprising the following residues:
Position 2 Val
Position 4 Leu
Position 20 Leu
Position 22 Cys
Position 24 Ala
Position 26 Gly
Position 29 Phe
Position 36 Trp
Position 47 Trp
Position 48 Ile
Position 69 Leu
Position 71 Thr
Position 78 Ala
Position 80 Met
Position 90 Tyr
Position 92 Cys
Position 94 Thr; and

viii) the light chain framework comprising the following residues:
Position 2 Ile
Position 3 Val
Position 4 Met
Position 23 Cys
Position 35 Trp
Position 36 Tyr
Position 46 Leu
Position 49 Tyr
Position 71 Phe
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 35, 36, and 37; or SEQ ID NOs: 38, 39, and 40, respectively.

In one embodiment, the present invention relates to an expression vector comprising a nucleotide sequence encoding a CDR sequence of an antibody selected from SEQ ID NO: 35, 36, 37, 38, 39, or 40.

In one embodiment, the present invention relates to an expression vector comprising nucleotide sequences encoding at least four CDR sequences of an antibody selected from the group consisting of SEQ ID NOs: 35, 36, 37, 38, 39, and 40.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 35, 36, and 37; and a second DNA sequence encoding at least the variable domain of the immunoglobulin light chain comprising CDR domains of SEQ ID NOs: 38, 39, and 40; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.

In one embodiment, an antibody comprises heavy and light chain variable regions comprising the amino acid sequences of SEQ ID NO:7 and SEQ ID NO:8, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:7 and SEQ ID NO:8, respectively.

In one embodiment, an antibody comprises CDR sequences of SEQ ID NOs: 41, 42, 43, 44, 45, and 46; or one or more of the CDR sequences can be conservative sequence modifications of the sequences SEQ ID NOs: 41, 42, 43, 44, 45, and 46.

In one embodiment, the present invention relates to an hybridoma which produces an antibody which comprises CDR sequences of SEQ ID NOs: 41, 42, 43, 44, 45, and 46.

In one embodiment, the present invention relates to a recombinant eukaryotic or prokaryotic cell which produces an antibody which comprises CDR sequences of SEQ ID NOs: 41, 42, 43, 44, 45, and 46.

In one embodiment, an antibody comprises at least one CDR sequence selected from (i) SEQ ID NO: 41, 42, 43, 44, 45, or 46; or (ii) a conservative sequence modification of the sequences listed in (i).

In one embodiment, an antibody comprises a polypeptide of SEQ ID NO:43.

In one embodiment, an antibody comprises at least four CDR sequences selected from the group consisting of SEQ ID NOs: 41, 42, 43, 44, 45, and 46; or one or more of the CDR sequences can be conservative sequence modifications of the sequences listed in SEQ ID NOs: 41, 42, 43, 44, 45, and 46.

In one embodiment, an antibody comprises heavy and light chain variable regions which comprise the CDR amino acid sequences of SEQ ID NOs: 41, 42, and 43, and SEQ ID NOs: 44, 45, and 46, respectively; or one or more of the CDR sequences can be conservative sequence modifications of the sequences listed in SEQ ID NOs: 41, 42, 43, 44, 45, and 46.

In one embodiment, an antibody of the present invention comprises:
i) CDRH1 as set out in SEQ ID NO. 41 or a variant of SEQ ID NO. 41 wherein Tyr32 is substituted for Ile, His, Phe, Thr, Asn, Cys, Glu or Asp and/or Asn33 is substituted for Gly, Tyr, Ala, Trp, Thr, Leu or Val and/or Ile34 is substituted for Met, Val or Trp and/or His35 is substituted for Ser, Glu, Asn, Gln, Tyr or Thr;
ii) CDRH2 as set out in SEQ ID NO. 42 or a variant of SEQ ID NO. 42 wherein Tyr50 is substituted for Arg, Glu, Trp, Gly, Gln, Val, Leu, Asn, Lys or Ala and/or Ile51 is substituted for Leu, Val, Thr, Ser or Asn and/or Asn52 is substituted for Asp, Leu, Ser or Tyr and/or Asn53 is substituted for Ala, Gly, Ser, Lys, Thr or Tyr and/or Ser54 is substituted for Asn, Thr, Lys, Asp or Gly and/or Gly56 is substituted for Tyr, Arg, Glu, Asp, Val, Ser or Ala and/or Gly58 is substituted for Lys, Asn, Ser, Asp, Arg, Thr, Phe or Tyr;
iii) CDRH3 as set out in SEQ ID NO. 43 or a variant of SEQ ID NO. 43 wherein Phe102 is substituted for Val, Tyr, His, Ile, Ser, Asp or Gly;
iv) CDRL1 as set out in SEQ ID NO. 44 or a variant of SEQ ID NO. 44 wherein Ser25 is substituted for Pro and/or Thr26 is substituted for Ser or Asn and/or Ser27A is substituted for SNDTE Asn, Asp, Thr or Glu and/or Ile27B is substituted for Leu and/or Val27C is substituted for Asp, Leu, Tyr, Ile, Ser, Asn, Phe, His, Gly or Thr and/or Pro27D is substituted for His or Leu and/or Asn28 is substituted for Asp or Ser and/or Thr31 is substituted for Ser, Asn, Lys or Gly and/or His32 is substituted for Phe, Tyr, Asn, Ala, Ser or Arg and/or Leu33 is substituted for Met, Val, Ile or Phe and/or G1u34 is substituted for His or Asn; and
v) CDRL3 as set out in SEQ ID NO. 46 or a variant of SEQ ID NO. 46 wherein Phe89 is substituted for Ser, Gly, Gln or Leu and/or Gln90 is substituted for His or Asn, Ala91 is substituted for Phe, Asn, Gly, Ser, Arg, Asp, His, Thr, Tyr or Val and/or Ser92 is substituted for Asn, Tyr, Thr, Trp, Arg, Gln, His, Ala or Asp and/or His93 is substituted for Glu, Asn, Gly, Thr, Ser, Arg or Ala and/or Val94 is substituted for Asp, Tyr, Thr, Leu, His, Asn, Ile, Trp, Pro or Ser and/or Trp96 is substituted for Pro, Leu, Tyr, Arg, Ile or Phe.

In one embodiment, antibody of the present invention comprises:

i) CDRH1 as set out in SEQ ID NO. 41

ii) CDRH2 as set out in SEQ ID NO. 42

iii) CDRH3 as set out in SEQ ID NO. 43

iv) CDRL1 as set out in SEQ ID NO. 44

v) CDRL2 as set out in SEQ ID NO. 45

vi) CDRL3 as set out in SEQ ID NO. 46;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Val, Ile, Phe, Leu or Ser
Position 36 Trp
Position 47 Trp or Tyr
Position 48 Ile, Met, Val or Leu
Position 69 Ile, Leu, Phe, Met or Val
Position 71 Ile, Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 71 Phe
Position 88 Cys
Position 98 Phe.

In one embodiment, an antibody of the present invention comprises:

i) CDRH3 as set out in SEQ ID NO. 43

ii) CDRH1 as set out in SEQ ID NO. 41

iii) CDRH2 as set out in SEQ ID NO. 42

iv) CDRL1 as set out in SEQ ID NO. 44

v) CDRL2 as set out in SEQ ID NO. 45

vi) CDRL3 as set out in SEQ ID NO. 46;

vii) the heavy chain framework comprising the following residues:
Position 2 Val
Position 4 Leu
Position 20 Met
Position 22 Cys
Position 24 Ala
Position 26 Gly
Position 29 Val
Position 36 Trp
Position 47 Trp
Position 48 Ile
Position 69 Leu
Position 71 Ile
Position 78 Ala
Position 80 Met
Position 90 Tyr
Position 92 Cys
Position 94 Arg; and

viii) the light chain framework comprising the following residues:
Position 2 Val
Position 3 Leu
Position 4 Met
Position 23 Cys
Position 35 Trp
Position 71 Phe
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 41, 42, and 43; or SEQ ID NOs: 44, 45, and 46, respectively.

In one embodiment, the present invention relates to an expression vector comprising a nucleotide sequence encoding a CDR sequence of an antibody selected from SEQ ID NO: 41, 42, 43, 44, 45, or 46.

In one embodiment, the present invention relates to an expression vector comprising nucleotide sequences encoding at least four CDR sequences of an antibody selected from the group consisting of SEQ ID NOs: 41, 42, 43, 44, 45, and 46.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 41, 42, and 43; and a second DNA sequence encoding at least the variable domain of the immunoglobulin light chain comprising CDR domains of SEQ ID NOs: 44, 45, and 46; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.

In one embodiment, an antibody comprises heavy and light chain variable regions comprising the amino acid sequences of SEQ ID NO:9 and SEQ ID NO:10, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:9 and SEQ ID NO:10, respectively.

In one embodiment, an antibody comprises CDR sequences of SEQ ID NOs: 47, 48, 49, 50, 51, and 52; or one or more of the CDR sequences can be conservative sequence modifications of the sequences SEQ ID NOs: 47, 48, 49, 50, 51, and 52.

In one embodiment, the present invention relates to an hybridoma which produces an antibody which comprises CDR sequences of SEQ ID NOs: 47, 48, 49, 50, 51, and 52.

In one embodiment, the present invention relates to a recombinant eukaryotic or prokaryotic cell which produces an antibody which comprises CDR sequences of SEQ ID NOs: 47, 48, 49, 50, 51, and 52.

In one embodiment, an antibody comprises at least one CDR sequence selected from (i) SEQ ID NO: 47, 48, 49, 50, 51, or 52; or (ii) a conservative sequence modification of the sequences listed in (i).

In one embodiment, an antibody comprises a polypeptide of SEQ ID NO:49.

In one embodiment, an antibody comprises at least four CDR sequences selected from the group consisting of SEQ ID NOs: 47, 48, 49, 50, 51, and 52; or one or more of the CDR sequences can be conservative sequence modifications of the sequences listed in SEQ ID NOs: 47, 48, 49, 50, 51, and 52.

In one embodiment, an antibody comprises heavy and light chain variable regions which comprise the CDR amino acid sequences of SEQ ID NOs: 47, 48, and 49, and SEQ ID NOs: 50, 51, and 52, respectively; or one or more of the CDR sequences can be conservative sequence modifications of the sequences listed in SEQ ID NOs: 47, 48, 49, 50, 51, and 52.

In one embodiment, an antibody of the present invention comprises:
i) CDRH1 as set out in SEQ ID NO. 47 or a variant of SEQ ID NO. 47 wherein Tyr32 is substituted for Ile, His, Phe, Thr, Asn, Cys, Glu or Asp and/or Tyr33 is substituted for Gly, Ala, Tip, Thr, Leu or Val and/or Met34 is substituted for Ile, Val or Tip and/or Asn35 is substituted for His, Glu, Ser, Gln, Tyr or Thr;
ii) CDRH2 as set out in SEQ ID NO. 48 or a variant of SEQ ID NO. 48 wherein Asp50 is substituted for Trp, Arg, Glu, Tyr, Gly, Gln, Val, Leu, Asn, Lys or Ala and/or Ile51 is substituted for Leu, Val, Thr, Ser or Asn and/or Asn52 is substituted for Asp, Leu, Ser or Tyr and/or Asn53 is substituted for Ala, Gly, Ser, Lys, Thr or Tyr and/or Asn54 is substituted for Ser, Thr, Lys, Asp or Gly and/or Asn56 is substituted for Val, Tyr, Arg, Glu, Asp, Gly, Ser or Ala and/or Asn58 is substituted for Lys, Thr, Ser, Asp, Arg, Gly, Phe or Tyr;
iii) CDRH3 as set out in SEQ ID NO. 49 or a variant of SEQ ID NO. 49 wherein Tyr102 is substituted for Val, His, Ile, Ser, Asp or Gly;
iv) CDRL1 as set out in SEQ ID NO. 50 or a variant of SEQ ID NO. 50 wherein Ser27A is substituted for Asn, Asp, Thr or Glu and/or Ser29 is substituted for Asp, Leu, Val, Ile, Tyr, Asn, Phe, His, Gly or Thr and/or Thr31 is substituted for Asn, Ser, Lys or Gly and/or Phe32 is substituted for Asn, Tyr, Ala, His, Ser or Arg and/or Leu33 is substituted for Met, Val, Ile or Phe; and
v) CDRL3 as set out in SEQ ID NO. 52 or a variant of SEQ ID NO. 52 wherein Gln89 is substituted for Ser, Gly, Phe or Leu and/or Gln90 is substituted for His or Asn, Tyr91 is substituted for Asn, Gly, Ser, Arg, Asp, His, Thr, Phe or Val and/or Ser92 is substituted for Asn, Tyr, Thr, Trp, Arg, Gln, His, Ala or Asp and/or Gly93 is substituted for Glu, Asn, Thr, His, Ser, Arg or Ala and/or Tyr94 is substituted for Asp, Thr, Val, Leu, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 47

ii) CDRH2 as set out in SEQ ID NO. 48

iii) CDRH3 as set out in SEQ ID NO. 49

iv) CDRL1 as set out in SEQ ID NO. 50

v) CDRL2 as set out in SEQ ID NO. 51

vi) CDRL3 as set out in SEQ ID NO. 52;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Asn, Ile, Leu or Val
Position 3 Val, Gln, Leu or Glu
Position 4 Met or Leu
Position 23 Cys
Position 35 Trp
Position 71 Tyr
Position 88 Cys
Position 98 Phe.

In one embodiment, an antibody of the present invention comprises:

i) CDRH3 as set out in SEQ ID NO. 49

ii) CDRH1 as set out in SEQ ID NO. 47

iii) CDRH2 as set out in SEQ ID NO. 48

iv) CDRL1 as set out in SEQ ID NO. 50

v) CDRL2 as set out in SEQ ID NO. 51

vi) CDRL3 as set out in SEQ ID NO. 52;

vii) the heavy chain framework comprising the following residues:
Position 2 Val
Position 4 Leu
Position 20 Ile
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Tyr
Position 92 Cys
Position 94 Gly; and

viii) the light chain framework comprising the following residues:
Position 2 Asn
Position 3 Val
Position 4 Leu
Position 23 Cys
Position 35 Trp
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 47, 48, and 49; or SEQ ID NOs: 50, 51, and 52, respectively.

In one embodiment, the present invention relates to an expression vector comprising a nucleotide sequence encoding a CDR sequence of an antibody selected from SEQ ID NO: 47, 48, 49, 50, 51, or 52.

In one embodiment, the present invention relates to an expression vector comprising nucleotide sequences encoding at least four CDR sequences of an antibody selected from the group consisting of SEQ ID NOs: 47, 48, 49, 50, 51, and 52.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 47, 48, and 49; and a second DNA sequence encoding at least the variable domain of the immunoglobulin light chain comprising CDR domains of SEQ ID NOs: 50, 51, and 52; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody comprises heavy and light chain variable regions comprising the amino acid sequences of SEQ ID NO:11 and SEQ ID NO:12, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:11 and SEQ ID NO:12, respectively.

In one embodiment, an antibody comprises CDR sequences of SEQ ID NOs: 53, 54, 55, 56, 57, and 58; or one or more of the CDR sequences can be conservative sequence modifications of the sequences SEQ ID NOs: 53, 54, 55, 56, 57, and 58.

In one embodiment, the present invention relates to an hybridoma which produces an antibody which comprises CDR sequences of SEQ ID NOs: 53, 54, 55, 56, 57, and 58. In one embodiment, the present invention relates to a recombinant eukaryotic or prokaryotic cell which produces an antibody which comprises CDR sequences of SEQ ID NOs: 53, 54, 55, 56, 57, and 58.

In one embodiment, an antibody comprises at least one CDR sequence selected from (i) SEQ ID NO: 53, 54, 55, 56, 57, or 58; or (ii) a conservative sequence modification of the sequences listed in (i).
In one embodiment, an antibody comprises a polypeptide of SEQ ID NO:55.

In one embodiment, an antibody comprises at least four CDR sequences selected from the group consisting of SEQ ID NOs: 53, 54, 55, 56, 57, and 58; or one or more of the CDR sequences can be conservative sequence modifications of the sequences listed in SEQ ID NOs: 53, 54, 55, 56, 57, and 58.

In one embodiment, an antibody comprises heavy and light chain variable regions which comprise the CDR amino acid sequences of SEQ ID NOs: 53, 54, and 55, and SEQ ID NOs: 56, 57, and 58, respectively; or one or more of the CDR sequences can be conservative sequence modifications of the sequences listed in SEQ ID NOs: 53, 54, 55, 56, 57, and 58.

In one embodiment, an antibody of the present invention comprises:
i) CDRH1 as set out in SEQ ID NO. 53 or a variant of SEQ ID NO. 53 wherein Tyr32 is substituted for Ile, His, Phe, Thr, Asn, Cys, Glu or Asp and/or Tyr33 is substituted for Gly, Ala, Trp, Thr, Leu or Val and/or Met34 is substituted for Ile, Val or Trp and/or Asn35 is substituted for His, Glu, Ser, Gln, Tyr or Thr;
ii) CDRH2 as set out in SEQ ID NO. 54 or a variant of SEQ ID NO. 54 wherein Asp50 is substituted for Trp, Arg, Glu, Tyr, Gly, Gln, Val, Leu, Asn, Lys or Ala and/or Ile51 is substituted for Leu, Val, Thr, Ser or Asn and/or Asn52 is substituted for Asp, Leu, Ser or Tyr and/or Asn53 is substituted for Ala, Gly, Ser, Lys, Thr or Tyr and/or Asn54 is substituted for Ser, Thr, Lys, Asp or Gly and/or Gly56 is substituted for Tyr, Arg, Glu, Asp, Val, Ser or Ala and/or Asn58 is substituted for Lys, Thr, Ser, Asp, Arg, Gly, Phe or Tyr;
iii) CDRH3 as set out in SEQ ID NO. 55 or a variant of SEQ ID NO. 55 wherein Tyr102 is substituted for Val, His, Ile, Ser, Asp or Gly;
iv) CDRL1 as set out in SEQ ID NO. 56 or a variant of SEQ ID NO. 56 wherein Ser27A is substituted for Asn, Asp, Thr or Glu and/or Ser29 is substituted for Asp, Leu, Val, Ile, Tyr, Asn, Phe, His, Gly or Thr and/or Thr31 is substituted for Asn, Ser, Lys or Gly and/or Tyr32 is substituted for Phe, Asn, Ala, His, Ser or Arg and/or Leu33 is substituted for Met, Val, Ile or Phe; and
v) CDRL3 as set out in SEQ ID NO. 58 or a variant of SEQ ID NO. 58 wherein Gln89 is substituted for Ser, Gly, Phe or Leu and/or Gln90 is substituted for His or Asn, Phe91 is substituted for Asn, Gly, Ser, Arg, Asp, His, Thr, Tyr or Val and/or Ser92 is substituted for Asn, Tyr, Thr, Trp, Arg, Gln, His, Ala or Asp and/or Gly93 is substituted for Glu, Asn, Thr, His, Ser, Arg or Ala and/or Tyr94 is substituted for Asp, Thr, Val, Leu, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 53

ii) CDRH2 as set out in SEQ ID NO. 54

iii) CDRH3 as set out in SEQ ID NO. 55

iv) CDRL1 as set out in SEQ ID NO. 56

v) CDRL2 as set out in SEQ ID NO. 57

vi) CDRL3 as set out in SEQ ID NO. 58;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Asn, Ile, Leu or Val
Position 3 Val, Gln, Leu or Glu
Position 4 Met or Leu
Position 23 Cys
Position 35 Trp
Position 71 Tyr
Position 88 Cys
Position 98 Phe.

In one embodiment, an antibody of the present invention comprises:

i) CDRH3 as set out in SEQ ID NO. 55

ii) CDRH1 as set out in SEQ ID NO. 53

iii) CDRH2 as set out in SEQ ID NO. 54

iv) CDRL1 as set out in SEQ ID NO. 56

v) CDRL2 as set out in SEQ ID NO. 57

vi) CDRL3 as set out in SEQ ID NO. 58;

vii) the heavy chain framework comprising the following residues:
Position 2 Val
Position 4 Leu
Position 20 Ile
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Phe
Position 92 Cys
Position 94 Gly

viii) the light chain framework comprising the following residues:
Position 2 Asn
Position 3 Val
Position 4 Leu
Position 23 Cys
Position 35 Trp
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 53, 54, and 55; or SEQ ID NOs: 56, 57, and 58, respectively.

In one embodiment, the present invention relates to an expression vector comprising a nucleotide sequence encoding a CDR sequence of an antibody selected from SEQ ID NO: 53, 54, 55, 56, 57, or 58.

In one embodiment, the present invention relates to an expression vector comprising nucleotide sequences encoding at least four CDR sequences of an antibody selected from the group consisting of SEQ ID NOs: 53, 54, 55, 56, 57, and 58.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 53, 54, and 55; and a second DNA sequence encoding at least the variable domain of the immunoglobulin light chain comprising CDR domains of SEQ ID NOs: 56, 57, and 58; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody has heavy and light chain variable regions comprising the amino acid sequences of SEQ ID NO:13 and SEQ ID NO:14, respectively, or conservative sequence modifications thereof.

In one embodiment, an antibody has heavy and light chain variable regions comprising polypeptides which are at least 90%, 95%, 98% or 99% identical to the amino acid sequences of SEQ ID NO:13 and SEQ ID NO:14, respectively.

In one embodiment, an antibody comprises CDR sequences of SEQ ID NOs: 59, 60, 61, 62, 63, and 64; or one or more of the CDR sequences can be conservative sequence modifications of the sequences SEQ ID NOs: 59, 60, 61, 62, 63, and 64.

In one embodiment, the present invention relates to an hybridoma which produces an antibody which comprises CDR sequences of SEQ ID NOs: 59, 60, 61, 62, 63, and 64.
In one embodiment, the present invention relates to a recombinant eukaryotic or prokaryotic cell which produces an antibody which comprises CDR sequences of SEQ ID NOs: 59, 60, 61, 62, 63, and 64.

In one embodiment, an antibody comprises at least one CDR sequence selected from (i) SEQ ID NO: 59, 60, 61, 62, 63, or 64; or (ii) a conservative sequence modification of the sequences listed in (i).

In one embodiment, an antibody comprises a polypeptide of SEQ ID NO:61.

In one embodiment, an antibody comprises at least four CDR sequences selected from the group consisting of SEQ ID NOs: 59, 60, 61, 62, 63, and 64; or one or more of the CDR sequences can be conservative sequence modifications of the sequences listed in SEQ ID NOs: 59, 60, 61, 62, 63, and 64.

In one embodiment, an antibody comprises heavy and light chain variable regions which comprise the CDR amino acid sequences of SEQ ID NOs: 59, 60, and 61, and SEQ ID NOs: 62, 63, and 64, respectively; or one or more of the CDR sequences can be conservative sequence modifications of the sequences listed in SEQ ID NOs: 59, 60, 61, 62, 63, and 64.

In one embodiment, an antibody of the present invention comprises:
i) CDRH1 as set out in SEQ ID NO. 59 or a variant of SEQ ID NO. 59 wherein Tyr32 is substituted for Ile, His, Phe, Thr, Asn, Cys, Glu or Asp and/or Trp33 is substituted for Tyr, Ala, Gly, Thr, Leu or Val and/or Met34 is substituted for Ile, Val or Trp and/or His35 is substituted for Ser, Glu, Asn, Gln, Tyr or Thr;
ii) CDRH2 as set out in SEQ ID NO. 60 or a variant of SEQ ID NO. 60 wherein Arg50 is substituted for Trp, Glu, Tyr, Gly, Gln, Val, Leu, Asn, Lys or Ala and/or Ile51 is substituted for Leu, Val, Thr, Ser or Asn and/or His52 is substituted for Asn, Asp, Leu, Ser or Tyr and/or Ser53 is substituted for Ala, Gly, Tyr, Lys, Thr or Asn and/or Asp54 is substituted for Asn, Thr, Lys, Ser or Gly and/or Asp56 is substituted for Tyr, Arg, Glu, Val, Gly, Ser or Ala and/or Asn58 is substituted for Lys, Thr, Ser, Asp, Arg, Gly, Phe or Tyr;
iii) CDRH3 as set out in SEQ ID NO. 61 or a variant of SEQ ID NO. 61 wherein Leu102 is substituted for Val, Tyr, His, Ile, Ser, Asp or Gly;
iv) CDRL1 as set out in SEQ ID NO. 62 or a variant of SEQ ID NO. 62 wherein Thr28 is substituted for Ser, Asp, Asn or Glu and/or Ile29 is substituted for Val and/or Gly30 is substituted for Asp, Leu, Val, Ile, Ser, Asn, Phe, His, Tyr or Thr and/or Thr31 is substituted for Asn, Ser, Lys or Gly and/or Trp32 is substituted for Asn, Phe, Tyr, Ala, His, Ser or Arg and/or Leu33 is substituted for Met, Val, Ile or Phe and/or Ala34 is substituted for Gly, Asn, Ser, His, Val or Phe;
v) CDRL2 as set out in SEQ ID NO. 63 or a variant of SEQ ID NO. 63 wherein Ala51 is substituted for Thr, Gly or Val; and
vi) CDRL3 as set out in SEQ ID NO. 64 or a variant of SEQ ID NO. 64 wherein Gln89 is substituted for Ser, Gly, Phe or Leu and/or Gln90 is substituted for His or Asn, Leu91 is substituted for Phe, Asn, Gly, Ser, Arg, Asp, His, Thr, Tyr or Val and/or Ser92 is substituted for Asn, Tyr, Thr, Trp, Arg, Gln, His, Ala or Asp and/or Ser93 is substituted for Glu, Asn, Gly, His, Thr, Arg or Ala and/or Thr94 is substituted for Asp, Tyr, Val, Leu, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 59

ii) CDRH2 as set out in SEQ ID NO. 60

iii) CDRH3 as set out in SEQ ID NO. 61

iv) CDRL1 as set out in SEQ ID NO. 62

v) CDRL2 as set out in SEQ ID NO. 63

vi) CDRL3 as set out in SEQ ID NO. 64;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Ile, Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe.

In one embodiment, an antibody of the present invention comprises:

i) CDRH3 as set out in SEQ ID NO. 61

ii) CDRH1 as set out in SEQ ID NO. 59

iii) CDRH2 as set out in SEQ ID NO. 60

iv) CDRL1 as set out in SEQ ID NO. 62

v) CDRL2 as set out in SEQ ID NO. 63

vi) CDRL3 as set out in SEQ ID NO. 64;

vii) the heavy chain framework comprises the following residues:
Position 2 Val
Position 4 Leu
Position 20 Val
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Tyr
Position 92 Cys
Position 94 Ile; and

viii) the light chain framework comprising the following residues:
Position 2 Ile
Position 3 Gln
Position 4 Met
Position 23 Cys
Position 35 Trp
Position 36 Tyr
Position 46 Leu
Position 49 Tyr
Position 71 Phe
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 59, 60, and 61; or SEQ ID NOs: 62, 63, and 64, respectively.

In one embodiment, the present invention relates to an expression vector comprising a nucleotide sequence encoding a CDR sequence of an antibody selected from SEQ ID NO: 59, 60, 61, 62, 63, or 64.

In one embodiment, the present invention relates to an expression vector comprising nucleotide sequences encoding at least four CDR sequences of an antibody selected from the group consisting of SEQ ID NOs: 59, 60, 61, 62, 63, and 64.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 59, 60, and 61; and a second DNA sequence encoding at least the variable domain of the immunoglobulin light chain comprising CDR domains of SEQ ID NOs: 62, 63, and 64; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody comprises heavy and light chain variable regions comprising the amino acid sequences of SEQ ID NO:15 and SEQ ID NO:16, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:15 and SEQ ID NO:16, respectively.

In one embodiment, an antibody comprises CDR sequences of SEQ ID NOs: 65, 66, 67, 68, 69, and 70; or one or more of the CDR sequences can be conservative sequence modifications of the sequences SEQ ID NOs: 65, 66, 67, 68, 69, and 70.

In one embodiment, the present invention relates to an hybridoma which produces an antibody which comprises CDR sequences of SEQ ID NOs: 65, 66, 67, 68, 69, or 70. In one embodiment, the present invention relates to a recombinant eukaryotic or prokaryotic cell which produces an antibody which comprises CDR sequences of SEQ ID NOs: 65, 66, 67, 68, 69, and 70.

In one embodiment, an antibody comprises at least one CDR sequence selected from (i) SEQ ID NO: 65, 66, 67, 68, 69, or 70; or (ii) a conservative sequence modification of the sequences listed in (i).

In one embodiment, an antibody comprises a polypeptide of SEQ ID NO:67.

In one embodiment, an antibody comprises at least four CDR sequences selected from the group consisting of SEQ ID NOs: 65, 66, 67, 68, 69, and 70; or one or more of the CDR sequences can be conservative sequence modifications of the sequences listed in SEQ ID NOs: 65, 66, 67, 68, 69, and 70.

In one embodiment, an antibody comprises heavy and light chain variable regions which comprise the CDR amino acid sequences of SEQ ID NOs: 65, 66, and 67, and SEQ ID NOs: 68, 69, and 70, respectively; or one or more of the CDR sequences can be conservative sequence modifications of the sequences listed in SEQ ID NOs: 65, 66, 67, 68, 69, and 70.

In one embodiment, an antibody of the present invention comprises:
i) CDRH1 as set out in SEQ ID NO. 65 or a variant of SEQ ID NO. 65 wherein Tyr32 is substituted for Ile, His, Phe, Thr, Asn, Cys, Glu or Asp and/or Asn33 is substituted for Gly, Tyr, Ala, Tip, Thr, Leu or Val and/or Met34 is substituted for Ile, Val or Trp and/or His35 is substituted for Ser, Glu, Asn, Gln, Tyr or Thr;
ii) CDRH2 as set out in SEQ ID NO. 66 or a variant of SEQ ID NO. 66 wherein Ala50 is substituted for Arg, Glu, Tyr, Gly, Gln, Val, Leu, Asn, Lys or Trp and/or Ile51 is substituted for Leu, Val, Thr, Ser or Asn and/or Tyr52 is substituted for Asp, Leu, Ser or Asn and/or Gly53 is substituted for Ala, Tyr, Ser, Lys, Thr or Asn and/or Asn54 is substituted for Ser, Thr, Lys, Asp or Gly and/or Asp56 is substituted for Tyr, Arg, Glu, Val, Gly, Ser or Ala and/or Ser58 is substituted for Lys, Asn, Thr, Asp, Arg, Gly, Phe or Tyr;
iii) CDRH3 as set out in SEQ ID NO. 67 or a variant of SEQ ID NO. 67 wherein Tyr102 is substituted for Val, His, Ile, Ser, Asp or Gly;
iv) CDRL1 as set out in SEQ ID NO. 68 or a variant of SEQ ID NO. 68 wherein Ser27A is substituted for Asn, Asp, Thr or Glu and/or Ser30 is substituted for Asp, Leu, Val, Ile, Tyr, Asn, Phe, His, Gly or Thr and/or Thr31 is substituted for Asn, Ser, Lys or Gly and/or Tyr32 is substituted for Phe, Asn, Ala, His, Ser or Arg and/or Leu33 is substituted for Met, Val, Ile or Phe; and
v) CDRL3 as set out in SEQ ID NO. 70 or a variant of SEQ ID NO. 70 wherein Gln89 is substituted for Ser, Gly, Phe or Leu and/or Gln90 is substituted for His or Asn, Phe91 is substituted for Asn, Gly, Ser, Arg, Asp, His, Thr, Tyr or Val and/or Ser92 is substituted for Asn, Tyr, Thr, Trp, Arg, Gln, His, Ala or Asp and/or Gly93 is substituted for Glu, Asn, Thr, His, Ser, Arg or Ala and/or Tyr94 is substituted for Asp, Thr, Val, Leu, His, Asn, Ile, Trp, Pro or Ser and/or Trp96 is substituted for Pro, Leu, Tyr, Arg, Ile or Phe.

In one embodiment, an antibody of the present invention comprises:

i) CDRH1 as set out in SEQ ID NO. 65

ii) CDRH2 as set out in SEQ ID NO. 66

iii) CDRH3 as set out in SEQ ID NO. 67

iv) CDRL1 as set out in SEQ ID NO. 68

v) CDRL2 as set out in SEQ ID NO. 69

vi) CDRL3 as set out in SEQ ID NO. 70;

vii) the heavy chain framework comprising the following residues:
Position 2 Ala, Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Asn, Ile, Leu or Val
Position 3 Val, Gln, Leu or Glu
Position 4 Met or Leu
Position 23 Cys
Position 35 Trp
Position 71 Tyr
Position 88 Cys
Position 98 Phe.

In one embodiment, an antibody of the present invention comprises:

i) CDRH3 as set out in SEQ ID NO. 67

ii) CDRH1 as set out in SEQ ID NO. 65

iii) CDRH2 as set out in SEQ ID NO. 66

iv) CDRL1 as set out in SEQ ID NO. 68

v) CDRL2 as set out in SEQ ID NO. 69

vi) CDRL3 as set out in SEQ ID NO. 70;

vii) the heavy chain framework comprising the following residues:
Position 2 Ala
Position 4 Leu
Position 20 Met
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Tyr
Position 92 Cys
Position 94 Arg; and

viii) the light chain framework comprising the following residues:
Position 2 Asn
Position 3 Val
Position 4 Leu
Position 23 Cys
Position 35 Trp
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 65, 66, and 67; or SEQ ID NOs: 68, 69, and 70, respectively.

In one embodiment, the present invention relates to an expression vector comprising a nucleotide sequence encoding a CDR sequence of an antibody selected from SEQ ID NO: 65, 66, 67, 68, 69, and 70.

In one embodiment, the present invention relates to an expression vector comprising nucleotide sequences encoding at least four CDR sequences of an antibody selected from the group consisting of SEQ ID NOs: 65, 66, 67, 68, 69, and 70.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 65, 66, and 67; and a second DNA sequence encoding at least the variable domain of the immunoglobulin light chain comprising CDR domains of SEQ ID NOs: 68, 69, and 70; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody comprises heavy and light chain variable regions comprising the amino acid sequences of SEQ ID NO:17 and SEQ ID NO:18, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:17 and SEQ ID NO:18, respectively.

In one embodiment, an antibody comprises CDR sequences of SEQ ID NOs: 71, 72, 73, 74, 75, and 76; or one or more of the CDR sequences can be conservative sequence modifications of the sequences SEQ ID NOs: 71, 72, 73, 74, 75, and 76.

In one embodiment, the present invention relates to an hybridoma which produces an antibody which comprises CDR sequences of SEQ ID NOs: 71, 72, 73, 74, 75, and 76. In one embodiment, the present invention relates to a recombinant eukaryotic or prokaryotic cell which produces an antibody which comprises CDR sequences of SEQ ID NOs: 71, 72, 73, 74, 75, and 76.

In one embodiment, an antibody comprises at least one CDR sequence selected from (i) SEQ ID NO: 71, 72, 73, 74, 75, or 76; or (ii) a conservative sequence modification of the sequences listed in (i).

In one embodiment, an antibody comprises a polypeptide of SEQ ID NO: 73.

In one embodiment, an antibody comprises at least four CDR sequences selected from the group consisting of SEQ ID NOs: 71, 72, 73, 74, 75, and 76; or one or more of the CDR sequences can be conservative sequence modifications of the sequences listed in SEQ ID NOs: 71, 72, 73, 74, 75, and 76.

In one embodiment, an antibody comprises heavy and light chain variable regions which comprise the CDR amino acid sequences of SEQ ID NOs: 71, 72, and 73, and SEQ ID NOs: 74, 75, and 76, respectively; or one or more of the CDR sequences can be conservative sequence modifications of the sequences listed in SEQ ID NOs: 71, 72, 73, 74, 75, and 76.

In one embodiment, an antibody of the present invention comprises:
i) CDRH1 as set out in SEQ ID NO. 71 or a variant of SEQ ID NO. 71 wherein Tyr32 is substituted for Ile, His, Phe, Thr, Asn, Cys, Glu or Asp and/or Gly33 is substituted for Tyr, Ala, Trp, Thr, Leu or Val and/or Ile34 is substituted for Met, Val or Trp and/or His35 is substituted for Ser, Glu, Asn, Gln, Tyr or Thr;
ii) CDRH2 as set out in SEQ ID NO. 72 or a variant of SEQ ID NO. 72 wherein Trp50 is substituted for Arg, Glu, Tyr, Gly, Gln, Val, Leu, Asn, Lys or Ala and/or Ile51 is substituted for Leu, Val, Thr, Ser or Asn and/or Asn52 is substituted for Asp, Leu, Ser or Tyr and/or Asn53 is substituted for Ala, Gly, Ser, Lys, Thr or Tyr and/or Thr54 is substituted for Asn, Ser, Lys, Asp or Gly and/or G1u56 is substituted for Tyr, Arg, Val, Asp, Gly, Ser or Ala and/or Thr58 is substituted for Lys, Asn, Ser, Asp, Arg, Gly, Phe or Tyr;
iii) CDRH3 as set out in SEQ ID NO. 73 or a variant of SEQ ID NO. 73 wherein Tyr102 is substituted for Val, His, Ile, Ser, Asp or Gly;
iv) CDRL1 as set out in SEQ ID NO. 74 or a variant of SEQ ID NO. 74 wherein Asn28 is substituted for Ser, Asp, Thr or Glu and/or Ile29 is substituted for Val and/or Tyr30 is substituted for Asp, Leu, Val, Ile, Ser, Asn, Phe, His, Gly or Thr and/or Ser31 is substituted for Asn, Thr, Lys or Gly and/or Asn32 is substituted for Phe, Tyr, Ala, His, Ser or Arg and/or Leu33 is substituted for Met, Val, Ile or Phe and/or Ala34 is substituted for Gly, Asn, Ser, His, Val or Phe;
v) CDRL2 as set out in SEQ ID NO. 75 or a variant of SEQ ID NO. 75 wherein Ala51 is substituted for Thr, Gly or Val; and
vi) CDRL3 as set out in SEQ ID NO. 76 or a variant of SEQ ID NO. 76 wherein Gln89 is substituted for Ser, Gly, Phe or Leu and/or His90 is substituted for Gln or Asn, Phe91 is substituted for Asn, Gly, Ser, Arg, Asp, His, Thr, Tyr or Val and/or Trp92 is substituted for Asn, Tyr, Thr, Ser, Arg, Gln, His, Ala or Asp and/or Gly93 is substituted for Glu, Asn, Thr, His, Ser, Arg or Ala and/or Thr94 is substituted for Asp, Tyr, Val, Leu, His, Asn, Ile, Trp, Pro or Ser and/or Leu96 is substituted for Pro, Trp, Tyr, Arg, Ile or Phe.

In one embodiment, an antibody of the present invention comprises:

i) CDRH1 as set out in SEQ ID NO. 71

ii) CDRH2 as set out in SEQ ID NO. 72

iii) CDRH3 as set out in SEQ ID NO. 73

iv) CDRL1 as set out in SEQ ID NO. 74

v) CDRL2 as set out in SEQ ID NO. 75

vi) CDRL3 as set out in SEQ ID NO. 76; and

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe.

In one embodiment, an antibody of the present invention comprises,

i) CDRH3 as set out in SEQ ID NO. 73

ii) CDRH1 as set out in SEQ ID NO. 71

iii) CDRH2 as set out in SEQ ID NO. 72

iv) CDRL1 as set out in SEQ ID NO. 74

v) CDRL2 as set out in SEQ ID NO. 75

vi) CDRL3 as set out in SEQ ID NO. 76;

vii) the heavy chain framework comprising the following residues:
Position 2 Ile
Position 4 Leu
Position 20 Ile
Position 22 Cys
Position 24 Ala
Position 26 Gly
Position 29 Leu
Position 36 Trp
Position 47 Trp
Position 48 Met
Position 69 Phe
Position 71 Leu
Position 78 Ala
Position 80 Leu
Position 90 Tyr
Position 92 Cys
Position 94 Lys; and

viii) the light chain framework comprising the following residues:
Position 2 Ile
Position 3 Gln
Position 4 Met
Position 23 Cys
Position 35 Trp
Position 36 Tyr
Position 46 Leu
Position 49 Tyr
Position 71 Phe
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 71, 72, and 73; or SEQ ID NOs: 74, 75, and 76, respectively.

In one embodiment, the present invention relates to an expression vector comprising a nucleotide sequence encoding a CDR sequence of an antibody selected from SEQ ID NO: 71, 72, 73, 74, 75, or 76.

In one embodiment, the present invention relates to an expression vector comprising nucleotide sequences encoding at least four CDR sequences of an antibody selected from the group consisting of SEQ ID NOs: 71, 72, 73, 74, 75, and 76.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 71, 72, and 73; and a second DNA sequence encoding at least the variable domain of the immunoglobulin light chain comprising CDR domains of SEQ ID NOs: 74, 75, and 76; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody comprises heavy and light chain variable regions comprising the amino acid sequences of SEQ ID NO:19 and SEQ ID NO: 20, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:19 and SEQ ID NO:20, respectively.

In one embodiment, an antibody comprises CDR sequences of SEQ ID NOs: 77, 78, 79, 80, 81, and 82; or one or more of the CDR sequences can be conservative sequence modifications of the sequences SEQ ID NOs: 77, 78, 79, 80, 81, and 82.

In one embodiment, the present invention relates to an hybridoma which produces an antibody which comprises CDR sequences of SEQ ID NOs: 77, 78, 79, 80, 81, and 82.
In one embodiment, the present invention relates to a recombinant eukaryotic or prokaryotic cell which produces an antibody which comprises CDR sequences of SEQ ID NOs: 77, 78, 79, 80, 81, and 82.

In one embodiment, an antibody comprises at least one CDR sequence selected from (i) SEQ ID NO: 77, 78, 79, 80, 81, or 82; or (ii) a conservative sequence modification of the sequences listed in (i).
In one embodiment, an antibody comprises a polypeptide of SEQ ID NO:79.

In one embodiment, an antibody comprises at least four CDR sequences selected from the group consisting of SEQ ID NOs: 77, 78, 79, 80, 81, and 82; or one or more of the CDR sequences can be conservative sequence modifications of the sequences listed in SEQ ID NOs: 77, 78, 79, 80, 81, and 82.

In one embodiment, an antibody comprises heavy and light chain variable regions which comprise the CDR amino acid sequences of SEQ ID NOs: 77, 78, and 79, and SEQ ID NOs: 80, 81, and 82, respectively; or one or more of the CDR sequences can be conservative sequence modifications of the sequences listed in SEQ ID NOs: 77, 78, 79, 80, 81, and 82.

In one embodiment, an antibody of the present invention comprises:
i) CDRH1 as set out in SEQ ID NO. 77 or a variant of SEQ ID NO. 77 wherein Asn32 is substituted for Ile, His, Phe, Thr, Tyr, Cys, Glu or Asp and/or Tyr33 is substituted for Gly, Ala, Trp, Thr, Leu or Val and/or Ile34 is substituted for Met, Val or Trp and/or Asp35 is substituted for Ser, His, Glu, Asn, Gln, Tyr or Thr;
ii) CDRH2 as set out in SEQ ID NO. 78 or a variant of SEQ ID NO. 78 wherein Trp50 is substituted for Arg, Glu, Tyr, Gly, Gln, Val, Leu, Asn, Lys or Ala and/or Ile51 is substituted for Leu, Val, Thr, Ser or Asn and/or Phe52 is substituted for Asn, Asp, Leu, Ser or Tyr and/or Gly53 is substituted for Ala, Tyr, Ser, Lys, Thr or Asn and/or Ser54 is substituted for Asn, Thr, Lys, Asp or Gly and/or Asn56 is substituted for Val, Tyr, Arg, Glu, Asp, Gly, Ser or Ala and/or Lys58 is substituted for Thr, Asn, Ser, Asp, Arg, Gly, Phe or Tyr;
iii) CDRH3 as set out in SEQ ID NO. 79 or a variant of SEQ ID NO. 79 wherein Val102 is substituted for Tyr, His, Ile, Ser, Asp or Gly;
iv) CDRL1 as set out in SEQ ID NO. 80 or a variant of SEQ ID NO. 80 wherein Asp28 is substituted for Ser, Asn, Thr or Glu and/or Val29 is substituted for Ile and/or Gly30 is substituted for Asp, Leu, Val, Ile, Ser, Asn, Phe, His, Tyr or Thr and/or Ser31 is substituted for Asn, Thr, Lys or Gly and/or Ala32 is substituted for Phe, Tyr, Asn, His, Ser or Arg and/or Val33 is substituted for Met, Leu, Ile or Phe and/or Ala34 is substituted for Gly, Asn, Ser, His, Val or Phe;
v) CDRL2 as set out in SEQ ID NO. 81 or a variant of SEQ ID NO. 81 wherein Ala51 is substituted for Thr, Gly or Val; and
vi) CDRL3 as set out in SEQ ID NO. 82 or a variant of SEQ ID NO. 82 wherein Gln89 is substituted for Ser, Gly, Phe or Leu and/or Gln90 is substituted for His or Asn, Tyr91 is substituted for Asn, Gly, Ser, Arg, Asp, His, Thr, Phe or Val and/or Ser92 is substituted for Asn, Tyr, Thr, Trp, Arg, Gln, His, Ala or Asp and/or Thr93 is substituted for Glu, Asn, Gly, His, Ser, Arg or Ala and/or Tyr94 is substituted for Asp, Thr, Val, Leu, His, Asn, Ile, Trp, Pro or Ser and/or Leu96 is substituted for Pro, Trp, Tyr, Arg, Ile or Phe.

In one embodiment, an antibody of the present invention comprises:

i) CDRH1 as set out in SEQ ID NO. 77

ii) CDRH2 as set out in SEQ ID NO. 78

iii) CDRH3 as set out in SEQ ID NO. 79

iv) CDRL1 as set out in SEQ ID NO. 80

v) CDRL2 as set out in SEQ ID NO. 81

vi) CDRL3 as set out in SEQ ID NO. 82;

vii) the heavy chain framework comprises the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp or Tyr
Position 48 Ile, Met, Val or Leu
Position 69 Ile, Leu, Phe, Met or Val
Position 71 Val, Ala or Leu
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprises the following residues:
Position 2 Ile, Leu or Val
Position 3 Val, Gln, Leu or Glu
Position 4 Met or Leu
Position 23 Ser or Cys
Position 35 Trp
Position 36 Tyr, Leu or Phe
Position 46 Leu, Arg or Val
Position 49 Tyr, His, Phe or Lys
Position 71 Tyr or Phe
Position 88 Cys
Position 98 Phe.

In one embodiment, an antibody of the present invention comprises:

i) CDRH3 as set out in SEQ ID NO. 79

ii) CDRH1 as set out in SEQ ID NO. 77

iii) CDRH2 as set out in SEQ ID NO. 78

iv) CDRL1 as set out in SEQ ID NO. 80

v) CDRL2 as set out in SEQ ID NO. 81

vi) CDRL3 as set out in SEQ ID NO. 82;

vii) the heavy chain framework comprising the following residues:
Position 2 Ile
Position 4 Leu
Position 20 Ile
Position 22 Cys
Position 24 Ala
Position 26 Gly
Position 29 Phe
Position 36 Trp
Position 47 Trp
Position 48 Ile
Position 69 Leu
Position 71 Val
Position 78 Ala
Position 80 Met
Position 90 Tyr
Position 92 Cys
Position 94 Arg; and

viii) the light chain framework comprising the following residues:
Position 2 Ile
Position 3 Val
Position 4 Met
Position 23 Ser
Position 35 Trp
Position 36 Tyr
Position 46 Leu
Position 49 Tyr
Position 71 Phe
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 77, 78, and 79; or SEQ ID NOs: 80, 81, and 82, respectively.

In one embodiment, the present invention relates to an expression vector comprising a nucleotide sequence encoding a CDR sequence of an antibody selected from SEQ ID NO: 77, 78, 79, 80, 81, or 82.

In one embodiment, the present invention relates to an expression vector comprising nucleotide sequences encoding at least four CDR sequences of an antibody selected from the group consisting of SEQ ID NOs: 77, 78, 79, 80, 81, and 82.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 77, 78, and 79; and a second DNA sequence encoding at least the variable domain of the immunoglobulin light chain comprising CDR domains of SEQ ID NOs: 80, 81, and 82; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.
   In one embodiment, an antibody comprises heavy and light chain variable regions comprising the amino acid sequences of SEQ ID NO:21 and SEQ ID NO: 22, respectively, or conservative sequence modifications thereof.

In one embodiment, an antibody comprises heavy and light chain variable regions comprising polypeptides which are at least 90%, 95%, 98% or 99% identical to the amino acid sequences of SEQ ID NO:21 and SEQ ID NO:22, respectively.

In one embodiment, an antibody comprises CDR sequences of SEQ ID NOs: 83, 84, 85, 86, 87, and 88; or one or more of the CDR sequences can be conservative sequence modifications of the sequences SEQ ID NOs: 83, 84, 85, 86, 87, and 88.

In one embodiment, the present invention relates to an hybridoma which produces an antibody which comprises CDR sequences of SEQ ID NOs: 83, 84, 85, 86, 87, and 88.
In one embodiment, the present invention relates to a recombinant eukaryotic or prokaryotic cell which produces an antibody which comprises CDR sequences of SEQ ID NOs: 83, 84, 85, 86, 87, and 88.

In one embodiment, an antibody comprises at least one CDR sequence selected from (i) SEQ ID NO: 83, 84, 85, 86, 87, or 88; or (ii) a conservative sequence modification of the sequences listed in (i).

In one embodiment, an antibody comprises a polypeptide of SEQ ID NO: 85.

In one embodiment, an antibody comprises at least four CDR sequences selected from the group consisting of SEQ ID NOs: 83, 84, 85, 86, 87, and 88; or one or more of the CDR sequences can be conservative sequence modifications of the sequences listed in SEQ ID NOs: 83, 84, 85, 86, 87, and 88.

In one embodiment, an antibody comprises heavy and light chain variable regions which comprise the CDR amino acid sequences of SEQ ID NOs: 83, 84, and 85, and SEQ ID NOs: 86, 87, and 88, respectively; or one or more of the CDR sequences can be conservative sequence modifications of the sequences listed in SEQ ID NOs: 83, 84, 85, 86, 87, and 88.

In one embodiment, an antibody of the present invention comprises:
i) CDRH1 as set out in SEQ ID NO. 83 or a variant of SEQ ID NO. 83 wherein Tyr32 is substituted for Ile, His, Phe, Thr, Asn, Cys, Glu or Asp and/or Tyr33 is substituted for Gly, Ala, Trp, Thr, Leu or Val and/or Met34 is substituted for Ile, Val or Trp and/or Tyr35 is substituted for His, Glu, Asn, Gln, Ser or Thr;
ii) CDRH2 as set out in SEQ ID NO. 84 or a variant of SEQ ID NO. 84 wherein Thr50 is substituted for Gly, Tyr, Phe, Ile, Glu or Val and/or Val51 is substituted for Leu, Ile, Thr, Ser or Asn and/or Ser52 is substituted for Phe, Trp or His and/or Val53 is substituted for Asp, Gly, Ser or Asn and/or Gly54 is substituted for Ser and/or Tyr56 is substituted for Ser, Thr, Asn, Asp or Arg and/or Lys58 is substituted for Asn, Thr, Ser, Asp, Arg, Gly, Phe or Tyr;
iii) CDRH3 as set out in SEQ ID NO. 85 or a variant of SEQ ID NO. 85 wherein Tyr102 is substituted for Val, His, Ile, Ser, Asp or Gly;
iv) CDRL1 as set out in SEQ ID NO. 86 or a variant of SEQ ID NO. 86 wherein Ser29 is substituted for Val, Asn, Asp, Thr or Glu and/or Val30 is substituted for Asp, Leu, Tyr, Ile, Ser, Asn, Phe, His, Gly or Thr and/or Ser31 is substituted for Asn, Thr, Lys or Gly and/or Tyr32 is substituted for Phe, Asn, Ala, His, Ser or Arg and/or Met33 is substituted for Leu, Val, Ile or Phe; and
v) CDRL3 as set out in SEQ ID NO. 88 or a variant of SEQ ID NO. 88 wherein His89 is substituted for Gln, Ser, Gly, Phe or Leu and/or Gln90 is substituted for His or Asn, Arg91 is substituted for Asn, Gly, Ser, Phe, Asp, His, Thr, Tyr or Val and/or Ser92 is substituted for Asn, Tyr, Thr, Trp, Arg, Gln, His, Ala or Asp and/or Ser93 is substituted for Tyr, Glu, Asn, Gly, His, Thr, Arg or Ala and/or Phe94 is substituted for Thr, Asp, Tyr, Val, Leu, His, Asn, Ile, Trp, Pro or Ser and/or Pro96 is substituted for Tip, Leu, Tyr, Arg, Ile or Phe.

In one embodiment, an antibody of the present invention comprises:

i) CDRH1 as set out in SEQ ID NO. 83

ii) CDRH2 as set out in SEQ ID NO. 84

iii) CDRH3 as set out in SEQ ID NO. 85

iv) CDRL1 as set out in SEQ ID NO. 86

v) CDRL2 as set out in SEQ ID NO. 87

vi) CDRL3 as set out in SEQ ID NO. 88;

vii) the heavy chain framework comprising the following residues:
Position 2 Val, Ile or Gly
Position 4 Leu or Val
Position 20 Leu, Ile, Met or Val
Position 22 Cys
Position 24 Thr, Ala, Val, Gly or Ser
Position 26 Gly
Position 29 Ile, Phe, Leu or Ser
Position 36 Trp
Position 47 Trp
Position 48 Ile, Met, Val or Leu
Position 69 Ile, Leu, Phe, Met or Val
Position 71 Arg
Position 78 Ala, Leu, Val, Tyr or Phe
Position 80 Leu or Met,
Position 90 Tyr or Phe
Position 92 Cys
Position 94 Arg, Lys, Gly, Ser, His or Asn; and

viii) the light chain framework comprising the following residues:
Position 2 Ile, Leu or Val
Position 3 Val, Gln, Leu or Glu
Position 4 Met or Leu
Position 23 Cys
Position 35 Trp
Position 71 Tyr
Position 88 Cys
Position 98 Phe.

In one embodiment, an antibody of the present invention comprises

i) CDRH3 as set out in SEQ ID NO. 85

ii) CDRH1 as set out in SEQ ID NO. 83

iii) CDRH2 as set out in SEQ ID NO. 84

iv) CDRL1 as set out in SEQ ID NO. 86

v) CDRL2 as set out in SEQ ID NO. 87

vi) CDRL3 as set out in SEQ ID NO. 88;

vii) the heavy chain framework comprising the following residues:
Position 2 Val
Position 4 Leu
Position 20 Leu
Position 22 Cys
Position 24 Ala
Position 26 Gly
Position 29 Phe
Position 36 Trp
Position 47 Trp
Position 48 Val
Position 69 Ile
Position 71 Arg
Position 78 Leu
Position 80 Leu
Position 90 Tyr
Position 92 Cys
Position 94 Arg; and

viii) the light chain framework comprising the following residues:
Position 2 Ile
Position 3 Val
Position 4 Leu
Position 23 Cys
Position 35 Trp
Position 71 Tyr
Position 88 Cys
Position 98 Phe.

In one embodiment, the present invention relates to an expression vector comprising nucleotide sequences encoding a variable heavy or light chain of an antibody comprising the CDR sequences of SEQ ID NOs: 83, 84, and 85; or SEQ ID NOs: 86, 87, and 88, respectively.

In one embodiment, the present invention relates to an expression vector comprising a nucleotide sequence encoding a CDR sequence of an antibody selected from SEQ ID NO: 83, 84, 85, 86, 87, or 88.

In one embodiment, the present invention relates to an expression vector comprising nucleotide sequences encoding at least four CDR sequences of an antibody selected from the group consisting of SEQ ID NOs: 83, 84, 85, 86, 87, and 88.

In one embodiment the present invention relates to a process for producing an antibody (immunoglobulin) in a single host cell, comprising the steps of:
(i) transforming said single host cell with a first DNA sequence encoding at least the variable domain of the immunoglobulin heavy chain comprising CDR domains of SEQ ID NOs: 83, 84, and 85; and a second DNA sequence encoding at least the variable domain of the immunoglobulin light chain comprising CDR domains of SEQ ID NOs: 86, 87, and 88; and
(ii) expressing said first DNA sequence and said second DNA sequence so that said immunoglobulin heavy and light chains are produced as separate molecules in said transformed single host cell;
   furthermore, this process can be carried out such that said first and second DNA sequences are present in different vectors or said first and second DNA sequences are present in a single vector.

In one embodiment the present invention relates to an antibody that fully or partially blocks the binding of any one of the aforementioned antibody to antigens selected from the group consisting of up to four human IL-8, Gro-alpha, Gro-beta, Gro-gamma, ENA-78, NAP2 and GCP-2 in an immunoassay, such as ELISA assay. In one embodiment, partial blocking occurs when the antibody blocks the binding of the antibody by more than 10%, 20%, 40% or 50%.

In one embodiment the present invention relates to an antibody that competes with the binding of any of the aforementioned antibody to antigens selected from the group consisting of up to four human IL-8, Gro-alpha, Gro-beta, Gro-gamma, ENA-78, NAP2 and GCP-2.
In one embodiment, the present invention relates to a composition comprising an aforementioned antibody and a pharmaceutically acceptable carrier.
In one embodiment, the present invention relates to a method of treating or preventing in a mammal COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury and/or endometriosis comprising administering an effective amount of an aforementioned antibody to said mammal.

In one embodiment the present invention relates to an aforementioned antibody for use in the treatment of diseases or disorders characterised by elevated or unbalanced level of one or more of human IL-8, Gro-alpha, Gro-beta, Gro-gamma, GCP-2 and ENA-78, particularly COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, or endometriosis.

In one aspect, the present invention relates to an aforementioned antibody for use in preventing and/or treating COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis in a mammal.

In one aspect, the present invention relates to use of an aforementioned antibody in the manufacture of a medicament for use in preventing and/or treating COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis in a mammal.

In one aspect, the present invention relates to use of an aforementioned antibody in the manufacture of a medicament for preventing and/or treating COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis in a mammal.

In one embodiment, above mammal is human.

### DESCRIPTION OF FIGURES

FIG. 1 depicts an exemplary set of MAPs to generate an antibody. For avoidance of doubt, five MAP peptide units are depicted. Each unit contains one identical amino acid sequence selected from linear peptides of SEQ ID NO: 89-93.

### DETAILED DESCRIPTION OF THE INVENTION

As used herein, “antibody” is also referred to as “immunoglobulin”. An antibody of the present invention is isolated. The term “antibody” is used herein in the broadest sense to refer to molecules with an immunoglobulin-like domain and includes monoclonal, recombinant, polyclonal, chimeric, humanised, bispecific and heteroconjugate antibodies; a single variable domain, a domain antibody, antigen binding fragments, immunologically effective fragments (such as Fab, F(ab′)2), single chain Fv, diabodies, Tandabs™, etc (for a summary of alternative “antibody” formats see Holliger and Hudson, Nature Biotechnology, 2005, Vol 23, No. 9, 1126-1136). In one embodiment, an antibody of this invention is monoclonal, humanized, chimeric, and immunologically effective fragments (such as Fab or F(ab′)2))

The phrase “single variable domain” refers to an antigen binding protein variable domain (for example, V_{H}, V_{HH}, V_{L}) that specifically binds an antigen or epitope independently of a different variable region or domain.

A “domain antibody” or “dAb” may be considered the same as a “single variable domain” which is capable of binding to an antigen. A single variable domain may be a human antibody variable domain, but also includes single antibody variable domains from other species such as rodent (for example, as disclosed in WO 00/29004), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are considered to be “domain antibodies”. As used herein V_{H} includes camelid V_{HH} domains.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain can bind an antigen or epitope independently of a different variable region or domain.

An antigen binding fragment may be provided by means of arrangement of one or more CDRs on non-antibody protein scaffolds such as a domain. A non-antibody protein scaffold or domain is one that has been subjected to protein engineering in order to obtain binding to a ligand other than its natural ligand, for example a domain which is a derivative of a scaffold selected from: CTLA-4 (Evibody); lipocalin; Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

CTLA-4 (Cytotoxic T Lymphocyte-associated Antigen 4) is a CD28-family receptor expressed on mainly CD4+T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001).

Lipocalins are a family of extracellular proteins which transport small hydrophobic molecules such as steroids, bilins, retinoids and lipids. They have a rigid β-sheet secondary structure with a number of loops at the open end of the canonical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to an antigen. The domain consists of a three-helical bundle of approximately 58 amino acids. Libraries have been generated by randomisation of surface residues. For further details see Protein Eng. Des. Sel. 17, 455-462 (2004) and EP1641818A1.

Avimers are multidomain proteins derived from the A-domain scaffold family. The native domains of approximately 35 amino acids adopt a defined disulphide bonded structure.

Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences, such as one or more CDRs, in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two a-helices and a β-turn. They can be engineered to bind different target antigens by: randomising residues in the first a-helix and a β-turn of each repeat; or insertion of peptide sequences, such as one or more CDRs. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges; examples of microproteins include KalataBl and conotoxin and knottins. The microproteins have a loop which can be engineered to include up to 25 amino acids without affecting the overall fold of the microprotein. For further details of engineered knottin domains, see WO2008098796.

Other binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Binding domains of the present invention could be derived from any of these alternative protein domains and any combination of the CDRs of the present invention grafted onto the domain.

An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

Throughout this specification, amino acid residue in variable domain sequences and full lengthantibody sequences are numbered according to the Kabat numbering convention, unless otherwise specified. For further information, see Kabat et al., Sequences of Proteins of Immunological Interest, 4^{th} Ed., U.S. Department of Health and Human Services, National Institutes of Health (1987).

As used herein, “an antibody that cross reacts with” means the antibody binds not only to one antigen but binds to other antigens as well. The antibodies of the present invention are isolated. By the term “up to four” means, one, two, three or four.

“Neutralizing,” as used herein is intended to refer to a partial or full inhibition of biological activities of up to four antigens selected from the group consisting of human IL-8, Gro-alpha, Gro-beta, Gro-gamma, GCP-2, NAP2, and ENA-78. For example, one of the biological activities of human IL-8, Gro-alpha, Gro-beta, Gro-gamma, NAP2, GCP-2, or ENA-78 is its ability to induce neutrophil chemotaxis.

One way of measuring the binding kinetics of an antibody is by surface plasmon resonance. The term “surface plasmon resonance”, as used herein, refers to an optical phenomenon that allows for the analysis of real-time biospecific interactions by detection of alterations in protein concentrations within a biosensor matrix, for example using the BIAcore system (GE Healthcare, Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics.

A “monoclonal antibody” or mAb (as opposed to polyclonal antibody) as used herein is intended to refer to a preparation of antibody molecules of single molecular composition. For example, a murine derived monoclonal antibody (mouse monoclonal antibody) can be prepared by hybridoma technology, such as the standard Kohler and Milstein hybridoma methodology.

Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol. 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36).

“Conservative sequence modifications” for nucleotide and amino acid sequence modifications means changes which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into the sequences by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an antibody for which sequence is specifically disclosed is preferably replaced with another amino acid residue from the same side chain family. Thus in one aspect, the antibody of the present invention includes all the conservative sequence modifications of the specifically disclosed amino acid sequences.

The present invention also encompasses “derivatives” of the amino acid sequences as specifically disclosed, wherein one or more of the amino acid residues have been derivatized, e.g., by acylation or glycosylation, without significantly affecting or altering the binding characteristics of the antibody containing the amino acid sequences.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial identity when the segments will hybridize under selective hybridization conditions, to the complement of the strand.

For nucleotide and amino acid sequences, the term “identity” indicates the degree of identity between two nucleic acid or amino acid sequences when optimally aligned and compared with appropriate insertions or deletions. Alternatively, substantial identity exists when the DNA segments will hybridize under selective hybridization conditions, to the complement of the strand.

The percent identity between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide or polypeptide sequences can be determined using the GAP program in the GCG software package (available at http://www.gcg.com), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

A nucleic acid is “operably linked” when it is placed into a functional relationship with another nucleic acid sequence. For instance, a promoter or enhancer is operably linked to a coding sequence if it affects the transcription of the sequence. With respect to transcription of regulatory sequences, operably linked means that the DNA sequences being linked are contiguous and where necessary to join two protein coding regions, contiguous and in reading frame. For switch sequences, operably linked indicates that the sequences are capable of effecting switch recombination.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell” or “recombinant cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell” as used herein. Recombinant host cells include, for example, transfectomas, such as CHO cells, NS/0 cells, and lymphocytic cells.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

### 1. Antibody Structures

### Intact Antibodies

Intact antibodies are usually heteromultimeric glycoproteins comprising at least two heavy and two light chains. Aside from IgM, intact antibodies are heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions. Each light chain has a variable domain (V_{L}) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1q component of the complement cascade. The human IgG2 constant region lacks the ability to activate complement by the classical pathway or to mediate antibody-dependent cellular cytotoxicity. The IgG4 constant region lacks the ability to activate complement by the classical pathway and mediates antibody-dependent cellular cytotoxicity only weakly. Antibodies essentially lacking these effector functions may be termed ‘non-lytic’ antibodies.

### Human Antibodies

Human antibodies may be produced by a number of methods known to those of skill in the art. Human antibodies can be made by the hybridoma method using human myeloma or mouse-human heteromyeloma cells lines see Kozbor J. Immunol 133, 3001, (1984) and Brodeur, Monoclonal Antibody Production Techniques and Applications, pp 51-63 (Marcel Dekker Inc, 1987). Alternative methods include the use of phage libraries or transgenic mice both of which utilize human V region repertories (see Winter G, (1994), Annu Rev. Immunol 12, 433-455, Green L L (1999), J. Immunol. methods 231, 11-23).

Several strains of transgenic mice are now available wherein their mouse immunoglobulin loci has been replaced with human immunoglobulin gene segments (see Tomizuka K, (2000) PNAS 97, 722-727; Fishwild D. M (1996) Nature Biotechnol. 14, 845-851, Mendez M J, 1997, Nature Genetics, 15, 146-156). Upon antigen challenge such mice are capable of producing a repertoire of human antibodies from which antibodies of interest can be selected.

Of particular note is the Trimera™ system (see Eren R et al, (1998) Immunology 93:154-161) where human lymphocytes are transplanted into irradiated mice, the Selected Lymphocyte Antibody System (SLAM, see Babcook et al, PNAS (1996) 93:7843-7848) where human (or other species) lymphocytes are effectively put through a massive pooled in vitro antibody generation procedure followed by deconvulated, limiting dilution and selection procedure and the Xenomouse II™ (Abgenix Inc). An alternative approach is available from Morphotek Inc using the Morphodoma™ technology.

Phage display technology can be used to produce human antibodies (and fragments thereof), see McCafferty; Nature, 348, 552-553 (1990) and Griffiths A D et at (1994) EMBO 13:3245-3260. According to this technique antibody V domain genes are cloned in frame into either a major or minor coat of protein gene of a filamentous bacteriophage such as M13 or fd and displayed (usually with the aid of a helper phage) as functional antibody fragments on the surface of the phage particle. Selections based on the functional properties of the antibody result in selection of the gene encoding the antibody exhibiting those properties. The phage display technique can be used to select antigen specific antibodies from libraries made from human B cells taken from individuals afflicted with a disease or disorder described above or alternatively from unimmunized human donors (see Marks; J. Mol. Bio. 222, 581-597, 1991). Where an intact human antibody is desired comprising a Fc domain it is necessary to redone the phage displayed derived fragment into a mammalian expression vectors comprising the desired constant regions and establishing stable expressing cell lines.

The technique of affinity maturation (Marks; Bio/technol 10, 779-783 (1992)) may be used to improve binding affinity wherein the affinity of the primary human antibody is improved by sequentially replacing the H and L chain V regions with naturally occurring variants and selecting on the basis of improved binding affinities. Variants of this technique such as “epitope imprinting” are now also available see WO 93/06213. See also Waterhouse; Nucl. Acids Res 21, 2265-2266 (1993).

### Chimaeric and Humanised Antibodies

The use of intact non-human antibodies in the treatment of human diseases or disorders carries with it the now well established problems of potential immunogenicity especially upon repeated administration of the antibody that is the immune system of the patient may recognise the non-human intact antibody as non-self and mount a neutralising response. In addition to developing fully human antibodies (see above) various techniques have been developed over the years to overcome these problems and generally involve reducing the composition of non-human amino acid sequences in the intact therapeutic antibody whilst retaining the relative ease in obtaining non-human antibodies from an immunised animal e.g. mouse, rat or rabbit. Broadly two approaches have been used to achieve this. The first are chimaeric antibodies, which generally comprise a non-human (e.g. rodent such as mouse) variable domain fused to a human constant region. Because the antigen-binding site of an antibody is localised within the variable regions the chimaeric antibody retains its binding affinity for the antigen but acquires the effector functions of the human constant region and are therefore able to perform effector functions such as described supra. Chimaeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chain variable regions of the antibody of the invention. Hybridoma cells serve as a typical source of such DNA. Once isolated, the DNA is placed into expression vectors which are then transfected into host cells such as E. coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions see e.g. Morrison; PNAS 81, 6851 (1984). Thus another embodiment of the invention there is provided a chimaeric antibody comprising a V_{H} domain having the sequence: SEQ ID No:2, 6, or 10 and a V_{L} domain having the sequence: SEQ ID No: 4, 8, or 12 fused to a human constant region (which maybe of a IgG isotype e.g. IgG1).

The second approach involves the generation of humanised antibodies wherein the non-human content of the antibody is reduced by humanizing the variable regions. Two techniques for humanisation have gained popularity. The first is humanisation by CDR grafting. CDRs build loops close to the antibody's N-terminus where they form a surface mounted in a scaffold provided by the framework regions. Antigen-binding specificity of the antibody is mainly defined by the topography and by the chemical characteristics of its CDR surface. These features are in turn determined by the conformation of the individual CDRs, by the relative disposition of the CDRs, and by the nature and disposition of the side chains of the residues comprising the CDRs. A large decrease in immunogenicity can be achieved by grafting only the CDRs of a non-human (e.g. murine) antibodies (“donor” antibodies) onto a suitable human framework (“acceptor framework”) and constant regions (see Jones et at (1986) Nature 321, 522-525 and Verhoeyen M et at (1988) Science 239, 1534-1536). However, CDR grafting per se may not result in the complete retention of antigen-binding properties and it is frequently found that some framework residues of the donor antibody need to be preserved (sometimes referred to as “backmutations”) in the humanised molecule if significant antigen-binding affinity is to be recovered (see Queen C et at (1989) PNAS 86, 10,029-10,033, Co, Met at (1991) Nature 351, 501-502). In this case, human V regions showing the greatest sequence homology (typically 60% or greater) to the non-human donor antibody maybe chosen from a database in order to provide the human framework (FR). The selection of human FRs can be made either from human consensus or individual human antibodies. Where necessary key residues from the donor antibody are substituted into the human acceptor framework to preserve CDR conformations. Computer modelling of the antibody maybe used to help identify such structurally important residues, see WO99/48523.

Alternatively, humanisation maybe achieved by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan E. A. et al; (1991) Mol. Immunol. 28, 489-498 and Pedersen J. T. et at (1994) J. Mol. Biol. 235; 959-973). Therefore it is possible to reduce the immunogenicity of a non-human Fv by replacing exposed residues in its framework regions that differ from those usually found in human antibodies. Because protein antigenicity can be correlated with surface accessibility, replacement of the surface residues may be sufficient to render the mouse variable region “invisible” to the human immune system (see also Mark G. E. et at (1994) in Handbook of Experimental Pharmacology vol. 113: The pharmacology of monoclonal Antibodies, Springer-Verlag, pp 105-134). This procedure of humanisation is referred to as “veneering” because only the surface of the antibody is altered, the supporting residues remain undisturbed. A further alternative approach is set out in WO04/006955. Further alternative approaches include that set out in WO04/006955 and the procedure of Humaneering™ (Kalobios) which makes use of bacterial expression systems and produces antibodies that are close to human germline in sequence (Alfenito-M Advancing Protein Therapeutics January 2007, San Diego, Calif.). Another, approach to humanisation involves selecting human acceptor frameworks on the basis of structural similarity of the human CDR regions to those of the donor mouse antibody CDR regions rather than on homology between other regions of the antibody such as framework regions. This process is also known as Superhumanisation™ (Evogenix Inc.; Hwang et al (2005) Methods 36:35-42).

It will be apparent to those skilled in the art that the term “derived” is intended to define not only the source in the sense of it being the physical origin for the material but also to define material which is structurally identical to the material but which does not originate from the reference source. Thus “residues found in the donor antibody” need not necessarily have been purified from the donor antibody.

### Antibody Fragments

In certain embodiments of the invention there is provided therapeutic antibody which is an antigen binding fragment. Such fragments may be functional antigen binding fragments of intact and/or humanised and/or chimaeric antibodies such as Fab, Fd, Fab′, F(ab′)₂, Fv, ScFv fragments of the antibodies described supra. Fragments lacking the constant region lack the ability to activate complement by the classical pathway or to mediate antibody-dependent cellular cytotoxicity. Traditionally such fragments are produced by the proteolytic digestion of intact antibodies by e.g. papain digestion (see for example, WO 94/29348) but may be produced directly from recombinantly transformed host cells. For the production of ScFv, see Bird et al; (1988) Science, 242, 423-426. In addition, antibody fragments may be produced using a variety of engineering techniques as described below.

Fv fragments appear to have lower interaction energy of their two chains than Fab fragments. To stabilise the association of the V_{H} and V_{L} domains, they have been linked with peptides (Bird et al, (1988) Science 242, 423-426, Huston et al, PNAS, 85, 5879-5883), disulphide bridges (Glockshuber et al, (1990) Biochemistry, 29, 1362-1367) and “knob in hole” mutations (Zhu et al (1997), Protein Sci., 6, 781-788). ScFv fragments can be produced by methods well known to those skilled in the art see Whitlow et al (1991) Methods companion Methods Enzymol, 2, 97-105 and Huston et al (1993) Int. Rev. Immunol 10, 195-217. ScFv may be produced in bacterial cells such as E. Coli but are more typically produced in eukaryotic cells. One disadvantage of ScFv is the monovalency of the product, which precludes an increased avidity due to polyvalent binding, and their short half-life. Attempts to overcome these problems include bivalent (ScFv')₂ produced from ScFV containing an additional C terminal cysteine by chemical coupling (Adams et at (1993) Can. Res 53, 4026-4034 and McCartney et at (1995) Protein Eng. 8, 301-314) or by spontaneous site-specific dimerization of ScFv containing an unpaired C terminal cysteine residue (see Kipriyanov et at (1995) Cell. Biophys 26, 187-204). Alternatively, ScFv can be forced to form multimers by shortening the peptide linker to between 3 to 12 residues to form “diabodies”, see Holliger et at PNAS (1993), 90, 6444-6448. Reducing the linker still further can result in ScFV trimers (“triabodies”, see Kortt et at (1997) Protein Eng, 10, 423-433) and tetramers (“tetrabodies”, see Le Gall et at (1999) FEBS Lett, 453, 164-168). Construction of bivalent ScFV molecules can also be achieved by genetic fusion with protein dimerizing motifs to form “miniantibodies” (see Pack et at (1992) Biochemistry 31, 1579-1584) and “minibodies” (see Hu et at (1996), Cancer Res. 56, 3055-3061). ScFv-Sc-Fv tandems ((ScFV)₂) may also be produced by linking two ScFv units by a third peptide linker, see Kurucz et at (1995) J. Immol. 154, 4576-4582. Bispecific diabodies can be produced through the noncovalent association of two single chain fusion products consisting of V_{H} domain from one antibody connected by a short linker to the V_{L} domain of another antibody, see Kipriyanov et at (1998), Int. J. Can 77, 763-772. The stability of such bispecific diabodies can be enhanced by the introduction of disulphide bridges or “knob in hole” mutations as described supra or by the formation of single chain diabodies (ScDb) wherein two hybrid ScFv fragments are connected through a peptide linker see Kontermann et at (1999) J. Immunol. Methods 226 179-188. Tetravalent bispecific molecules are available by e.g. fusing a ScFv fragment to the CH3 domain of an IgG molecule or to a Fab fragment through the hinge region see Coloma et at (1997) Nature Biotechnol. 15, 159-163. Alternatively, tetravalent bispecific molecules have been created by the fusion of bispecific single chain diabodies (see Alt et al, (1999) FEBS Lett 454, 90-94. Smaller tetravalent bispecific molecules can also be formed by the dimerization of either ScFv-ScFv tandems with a linker containing a helix-loop-helix motif (DiBi miniantibodies, see Muller et at (1998) FEBS Lett 432, 45-49) or a single chain molecule comprising four antibody variable domains (V_{H} and V_{L}) in an orientation preventing intramolecular pairing (tandem diabody, see Kipriyanov et al, (1999) J. Mol. Biol. 293, 41-56). Bispecific F(ab′)2 fragments can be created by chemical coupling of Fab′ fragments or by heterodimerization through leucine zippers (see Shalaby et al, (1992) J. Exp. Med. 175, 217-225 and Kostelny et at (1992), J. Immunol. 148, 1547-1553). Also available are so-called domain antibodies based on isolated V_{H} or V_{L} domains (Domantis Ltd.), see U.S. Pat. No. 6,248,516; U.S. Pat. No. 6,291,158; U.S. Pat. No. 6,172,197.

### Heteroconjugate Antibodies

Heteroconjugate antibodies are derivatives which also form an embodiment of the present invention. Heteroconjugate antibodies are composed of two covalently joined antibodies formed using any convenient cross-linking methods. See U.S. Pat. No. 4,676,980.

### Other Modifications.

Antibodies of the present invention may also incorporate any other modifications in the constant regions. For example glycosylation of antibodies at conserved positions in their constant regions is known to have a profound effect on antibody function, particularly effector functioning such as those described above, see for example, Boyd et at (1996), Mol. Immunol. 32, 1311-1318. Glycosylation variants of the therapeutic antibodies of the present invention wherein one or more carbohydrate moiety is added, substituted, deleted or modified are contemplated. Introduction of an asparagine-X-serine or asparagine-X-threonine motif creates a potential site for enzymatic attachment of carbonhydrate moieties and may therefore be used to manipulate the glycosylation of an antibody. In Raju et at (2001) Biochemistry 40, 8868-8876 the terminal sialyation of a TNFR-IgG immunoadhesin was increased through a process of regalactosylation and/or resialylation using beta-1,4-galactosyltransferace and/or alpha, 2,3 sialyltransferase. Increasing the terminal sialylation is believed to increase the half-life of the immunoglobulin. Antibodies, in common with most glycoproteins, are typically produced in nature as a mixture of glycoforms. This mixture is particularly apparent when antibodies are produced in eukaryotic, particularly mammalian cells. A variety of methods have been developed to manufacture defined glycoforms, see Zhang et at Science (2004), 303, 371, Sears et al, Science, (2001) 291, 2344, Wacker et al, (2002) Science, 298 1790, Davis et at (2002) Chem. Rev. 102, 579, Hang et at (2001) Acc. Chem. Res 34, 727. Thus the invention concerns a plurality of therapeutic antibodies (which maybe of the IgG isotype, e.g. IgG1) as described herein comprising a defined number (e.g. 7 or less, for example 5 or less such as two or a single) glycoform(s) of said antibodies.

Derivatives according to the invention also include therapeutic antibodies of the invention coupled to a non-proteinaeous polymer such as polyethylene glycol (PEG), polypropylene glycol or polyoxyalkylene. Conjugation of proteins to PEG is an established technique for increasing half-life of proteins, as well as reducing antigenicity and immunogenicity of proteins. The use of PEGylation with different molecular weights and styles (linear or branched) has been investigated with intact antibodies as well as Fab′ fragments, see Koumenis I. L. et at (2000) Int. J. Pharmaceut. 198:83-95. A particular embodiment comprises an antigen-binding fragment of the invention without the effector functions of a) activation of complement by the classical pathway; and b) mediating antibody-dependent cellular cytotoxicity; (such as a Fab fragment or a scFv) coupled to PEG.

The interaction between the Fc region of an antibody and various Fc receptors (FcγR) is believed to mediate the effector functions of the antibody which include antibody-dependent cellular cytotoxicity (ADCC), fixation of complement, phagocytosis and half-life/clearance of the antibody. Various modifications to the Fc region of antibodies of the invention may be carried out depending on the desired effector property. In particular, human constant regions which essentially lack the functions of a) activation of complement by the classical pathway; and b) mediating antibody-dependent cellular cytotoxicity include the IgG4 constant region, the IgG2 constant region and IgG1 constant regions containing specific mutations as for example mutations at positions 234, 235, 236, 237, 297, 318, 320 and/or 322 disclosed in EP0307434 (WO8807089), EP 0629 240 (WO9317105) and WO 2004/014953. Mutations at residues 235 or 237 within the CH2 domain of the heavy chain constant region (Kabat numbering; EU Index system) have separately been described to reduce binding to FcγRI, FcγRII and FcγRIII binding and therefore reduce antibody-dependent cellular cytotoxicity (ADCC) (Duncan et al. Nature 1988, 332; 563-564; Lund et al. J. Immunol. 1991, 147; 2657-2662; Chappel et al. PNAS 1991, 88; 9036-9040; Burton and Woof, Adv. Immunol. 1992, 51; 1-84; Morgan et al., Immunology 1995, 86; 319-324; Hezareh et al., J. Virol. 2001, 75 (24); 12161-12168). Further, some reports have also described involvement of some of these residues in recruiting or mediating complement dependent cytotoxicity (CDC) (Morgan et al., 1995; Xu et al., Cell. Immunol. 2000; 200:16-26; Hezareh et al., J. Virol. 2001, 75 (24); 12161-12168). Residues 235 and 237 have therefore both been mutated to alanine residues (Brett et al. Immunology 1997, 91; 346-353; Bartholomew et al. Immunology 1995, 85; 41-48; and WO9958679) to reduce both complement mediated and FcγR-mediated effects. Antibodies comprising these constant regions may be termed ‘non-lytic’ antibodies.

One may incorporate a salvage receptor binding epitope into the antibody to increase serum half life see U.S. Pat. No. 5,739,277.

There are five currently recognised human Fcγ receptors, FcγR (I), FcγRIIa, FcγRIIb, FcγRIIIa and neonatal FcRn. Shields et al, (2001) J. Biol. Chem. 276, 6591-6604 demonstrated that a common set of IgG1 residues is involved in binding all FcγR5, while FcγRII and FcγRIII utilize distinct sites outside of this common set. One group of IgG1 residues reduced binding to all FcγR5 when altered to alanine: Pro-238, Asp-265, Asp-270, Asn-297 and Pro-239. All are in the IgG CH2 domain and clustered near the hinge joining CH1 and CH2. While FcγRI utilizes only the common set of IgG1 residues for binding, FcγRII and FcγRIII interact with distinct residues in addition to the common set. Alteration of some residues reduced binding only to FcγRII (e.g. Arg-292) or FcγRIII (e.g. Glu-293). Some variants showed improved binding to FcγRII or FcγRIII but did not affect binding to the other receptor (e.g. Ser-267Ala improved binding to FcγRII but binding to FcγRIII was unaffected). Other variants exhibited improved binding to FcγRII or FcγRIII with reduction in binding to the other receptor (e.g. Ser-298Ala improved binding to FcγRIII and reduced binding to FcγRII). For FcγRIIIa, the best binding IgG1 variants had combined alanine substitutions at Ser-298, Glu-333 and Lys-334. The neonatal FcRn receptor is believed to be involved in protecting IgG molecules from degradation and thus enhancing serum half life and the transcytosis across tissues (see Junghans R. P (1997) Immunol. Res 16. 29-57 and Ghetie et at (2000) Annu Rev. Immunol. 18, 739-766). Human IgG1 residues determined to interact directly with human FcRn includes Ile253, Ser254, Lys288, Thr307, Gln311, Asn434 and His435.

The therapeutic antibody of the invention may incorporate any of the above constant region modifications.

### 2. Production Methods

Antibodies of the present invention may be produced in transgenic organisms such as goats (see Pollock et at (1999), J. Immunol. Methods 231:147-157), chickens (see Morrow K J J (2000) Genet. Eng. News 20:1-55), mice (see Pollock et al, ibicl) or plants (see Doran P M, (2000) Curr. Opinion Biotechnol. 11, 199-204, Ma J K-C (1998), Nat. Med. 4; 601-606, Baez J et al, BioPharm (2000) 13: 50-54, Stoger E et al; (2000) Plant Mol. Biol. 42:583-590). Antibodies may also be produced by chemical synthesis. However, antibodies of the invention are typically produced using recombinant cell culturing technology well known to those skilled in the art. A polynucleotide encoding the antibody is isolated and inserted into a replicable vector such as a plasmid for further cloning (amplification) or expression in a host cell. One useful expression system is a glutamate synthetase system (such as sold by Lonza Biologics), particularly where the host cell is CHO or NS0 (see below). Polynucleotide encoding the antibody is readily isolated and sequenced using conventional procedures (e.g. oligonucleotide probes). Vectors that may be used include plasmid, virus, phage, transposons, minichromsomes of which plasmids are a typical embodiment. Generally such vectors further include a signal sequence, origin of replication, one or more marker genes, an enhancer element, a promoter and transcription termination sequences operably linked to the light and/or heavy chain polynucleotide so as to facilitate expression. Polynucleotide encoding the light and heavy chains may be inserted into separate vectors and introduced (e.g. by transformation, transfection, electroporation or transduction) into the same host cell concurrently or sequentially or, if desired both the heavy chain and light chain can be inserted into the same vector prior to such introduction.

It will be immediately apparent to those skilled in the art that due to the redundancy of the genetic code, alternative polynucleotides to those disclosed herein are also available that will encode the polypeptides of the invention.

### Signal Sequences

Antibodies of the present invention maybe produced as a fusion protein with a heterologous signal sequence having a specific cleavage site at the N terminus of the mature protein. The signal sequence should be recognised and processed by the host cell. For prokaryotic host cells, the signal sequence may be an alkaline phosphatase, penicillinase, or heat stable enterotoxin II leaders. For yeast secretion the signal sequences may be a yeast invertase leader, a factor leader or acid phosphatase leaders see e.g. WO90/13646. In mammalian cell systems, viral secretory leaders such as herpes simplex gD signal and a native immunoglobulin signal sequence (such as human Ig heavy chain) are available. Typically the signal sequence is ligated in reading frame to polynucleotide encoding the antibody of the invention.

### Origin of Replication

Origin of replications are well known in the art with pBR322 suitable for most gram-negative bacteria, 2u plasmid for most yeast and various viral origins such as SV40, polyoma, adenovirus, VSV or BPV for most mammalian cells. Generally the origin of replication component is not needed for integrated mammalian expression vectors, unless vector propagation is required in E. coli. However the SV40 on may be used since it contains the early promoter.

### Selection Marker

Typical selection genes encode proteins that (a) confer resistance to antibiotics or other toxins e.g. ampicillin, neomycin, methotrexate or tetracycline or (b) complement auxotrophic deficiencies or supply nutrients not available in the complex media or (c) combinations of both. The selection scheme may involve arresting growth of the host cells that contain no vector or vectors. Cells, which have been successfully transformed with the genes encoding the therapeutic antibody of the present invention, survive due to e.g. drug resistance conferred by the co-delivered selection marker. One example is the DHFR-selection system wherein transformants are generated in DHFR negative host strains (eg see Page and Sydenham 1991 Biotechnology 9: 64-68). In this system the DHFR gene is co-delivered with antibody polynucleotide sequences of the invention and DHFR positive cells then selected by nucleoside withdrawal. If required, the DHFR inhibitor methotrexate is also employed to select for transformants with DHFR gene amplification. By operably linking DHFR gene to the antibody coding sequences of the invention or functional derivatives thereof, DHFR gene amplification results in concomitant amplification of the desired antibody sequences of interest. CHO cells are a particularly useful cell line for this DHFR/methotrexate selection and methods of amplifying and selecting host cells using the DHFR system are well established in the art see Kaufman R. J. et al J. Mol. Biol. (1982) 159, 601-621, for review, see Werner R G, Noe W, Kopp K, Schluter M,” Appropriate mammalian expression systems for biopharmaceuticals”, Arzneimittel-Forschung. 48(8):870-80, 1998 August A further example is the glutamate synthetase expression system (Lonza Biologics). A suitable selection gene for use in yeast is the trp1 gene; see Stinchcomb et at Nature 282, 38, 1979.

### Promoters

Suitable promoters for expressing antibodies of the invention are operably linked to DNA/polynucleotide encoding the antibody. Promoters for prokaryotic hosts include phoA promoter, Beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase, among others. Inducible yeast promoters include alcohol dehydrogenase 2, isocytochrome C, acid phosphatase, metallothionein and enzymes responsible for nitrogen metabolism or maltose/galactose utilization, among others.

Promoters for expression in mammalian cell systems include RNA polymerase II promoters including viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40 and non-viral promoters such as EF-1alpha (Mizushima and Nagata Nucleic Acids Res 1990 18(17):5322, among others. The choice of promoter may be based upon suitable compatibility with the host cell used for expression.

### Enhancer Element

Where appropriate, e.g. for expression in higher eukaroytics, additional enhancer elements can included instead of or as well as those found located in the promoters described above. Suitable mammalian enhancer sequences include enhancer elements from globin, elastase, albumin, fetoprotein, metallothionine and insulin. Alternatively, one may use an enhancer element from a eukaroytic cell virus such as SV40 enhancer, cytomegalovirus early promoter enhancer, polyoma enhancer, baculoviral enhancer or murine IgG2a locus (see WO04/009823). Whilst such enhancers are typically located on the vector at a site upstream to the promoter, they can also be located elsewhere e.g. within the untranslated region or downstream of the polydenalytion signal. The choice and positioning of enhancer may be based upon suitable compatibility with the host cell used for expression.

### Polyadenylation/Termination

In eukaryotic systems, polyadenylation signals are operably linked to polynucleotide encoding the antibody of this invention. Such signals are typically placed 3′ of the open reading frame. In mammalian systems, non-limiting example signals include those derived from growth hormones, elongation factor-1 alpha and viral (eg SV40) genes or retroviral long terminal repeats. In yeast systems non-limiting examples of polydenylation/termination signals include those derived from the phosphoglycerate kinase (PGK) and the alcohol dehydrogenase 1 (ADH) genes. In prokaryotic system polyadenylation signals are typically not required and it is instead usual to employ shorter and more defined terminator sequences. The choice of polyadenylation/termination sequences may be based upon suitable compatibility with the host cell used for expression,

### Other Methods/Elements for Enhanced Yields

In addition to the above, other features that can be employed to enhance yields include chromatin remodelling elements, introns and host-cell specific codon modification. The codon usage of the antibody of this invention thereof can be modified to accommodate codon bias of the host cell such to augment transcript and/or product yield (eg Hoekema A et al Mol Cell Biol 1987 7(8):2914-24). The choice of codons may be based upon suitable compatibility with the host cell used for expression.

### Host Cells

Suitable host cells for cloning or expressing vectors encoding antibodies of the invention are, for example, prokaroytic, yeast or higher eukaryotic cells. Suitable prokaryotic cells include eubacteria e.g. enterobacteriaceae such as Escherichia e.g. E. coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. licheniformis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et al J. Biotechnol. 108 (2004) 185-192), Candida, Trichoderma reesia (EP244, 234), Penicillin, Tolypocladium and Aspergillus hosts such as A. nidulans and A. niger are also contemplated.

Although Prokaryotic and yeast host cells are specifically contemplated by the invention, typically however, host cells of the present invention are vertebrate cells. Suitable vertebrate host cells include mammalian cells such as COS-1 (ATCC No. CRL 1650) COS-7 (ATCC CRL 1651), human embryonic kidney line 293, PerC6 (Crucell), baby hamster kidney cells (BHK) (ATCC CRL. 1632), BHK570 (ATCC NO: CRL 10314), 293 (ATCC NO. CRL 1573), Chinese hamster ovary cells CHO (e.g. CHO-K1, ATCC NO: CCL 61, DHFR-CHO cell line such as DG44 (see Urlaub et al, (1986) ibid), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NS0 (see U.S. Pat. No. 5,807,715), Sp2/0, Y0.

Thus in one embodiment of the invention there is provided a stably transformed host cell comprising a vector encoding a heavy chain and/or light chain of the therapeutic antibody as described herein. Typically such host cells comprise a first vector encoding the light chain and a second vector encoding said heavy chain.

Such host cells may also be further engineered or adapted to modify quality, function and/or yield of the antibody of this invention. Non-limiting examples include expression of specific modifying (eg glycosylation) enzymes and protein folding chaperones.

### Cell Culturing Methods.

Host cells transformed with vectors encoding the therapeutic antibodies of the invention may be cultured by any method known to those skilled in the art. Host cells may be cultured in spinner flasks, shake flasks, roller bottles or hollow fibre systems but it is preferred for large scale production that stirred tank reactors or bag reactors (eg Wave Biotech, Somerset, N.J. USA) are used particularly for suspension cultures. Typically the stirred tankers are adapted for aeration using e.g. spargers, baffles or low shear impellers. For bubble columns and airlift reactors direct aeration with air or oxygen bubbles maybe used. Where the host cells are cultured in a serum free culture media it is preferred that the media is supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly vertebrate host cells may utilise a variety of operational modes such as batch, fed-batch, repeated batch processing (see Drapeau et at (1994) cytotechnology 15: 103-109), extended batch process or perfusion culture. Although recombinantly transformed mammalian host cells may be cultured in serum-containing media such media comprising fetal calf serum (FCS), it is preferred that such host cells are cultured in synthetic serum-free media such as disclosed in Keen et at (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et at (1995) in Animal Cell technology: Developments towards the 21st century (Beuvery E. C. et at eds), pp 619-623, Kluwer Academic publishers).

Antibodies of the invention secreted into the media may be recovered and purified from the media using a variety of techniques to provide a degree of purification suitable for the intended use. For example the use of therapeutic antibodies of the invention for the treatment of human patients typically mandates at least 95% purity as determined by reducing SDS-PAGE, more typically 98% or 99% purity, when compared to the culture media comprising the therapeutic antibodies. In the first instance, cell debris from the culture media is typically removed using centrifugation followed by a clarification step of the supernatant using e.g. microfiltration, ultrafiltration and/or depth filtration. Alternatively, the antibody can be harvested by microfiltration, ultrafiltration or depth filtration without prior centrifugation. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. In one embodiment, the antibodies of the invention, following various clarification steps, are captured using Protein A or G affinity chromatography followed by further chromatography steps such as ion exchange and/or HA chromatography, anion or cation exchange, size exclusion chromatography and ammonium sulphate precipitation. Typically, various virus removal steps are also employed (e.g. nanofiltration using e.g. a DV-20 filter). Following these various steps, a purified (typically monoclonal) preparation comprising at least 10 mg/ml or greater e.g. 100 mg/ml or greater of the antibody of the invention is provided and therefore forms an embodiment of the invention. Concentration to 100 mg/ml or greater can be generated by ultracentrifugation. Suitably such preparations are substantially free of aggregated forms of antibodies of the invention.

Bacterial systems are particularly suited for the expression of antibody fragments. Such fragments are localised intracellularly or within the periplasma. Insoluble periplasmic proteins can be extracted and refolded to form active proteins according to methods known to those skilled in the art, see Sanchez et at (1999) J. Biotechnol. 72, 13-20 and Cupit P M et at (1999) Lett Appl Microbiol, 29, 273-277.

### 3. Pharmaceutical Compositions and Mode of Administration

Purified preparations of antibodies of the invention as described supra, may be incorporated into pharmaceutical compositions for use in the treatment of human diseases and disorders such as those outlined above. Typically such compositions further comprise a pharmaceutically acceptable (i.e. inert) carrier as known and called for by acceptable pharmaceutical practice, see e.g. Remingtons Pharmaceutical Sciences, 16th ed, (1980), Mack Publishing Co. Examples of such carriers include sterilised carrier such as saline, Ringers solution or dextrose solution, buffered with suitable buffers to a pH within a range of 5 to 8. Pharmaceutical compositions for injection (e.g. by intravenous, intraperitoneal, intradermal, subcutaneous, intramuscular or intraportal) or continuous infusion are suitably free of visible particulate matter and may comprise from 0.1 mg to 10 g of therapeutic antibody, typically between 5 mg and 25 mg of antibody. Methods for the preparation of such pharmaceutical compositions are well known to those skilled in the art. In one embodiment, pharmaceutical compositions comprise from 0.1 mg to 10 g of therapeutic antibodies of the invention in unit dosage form, optionally together with instructions for use. Pharmaceutical compositions of the invention may be lyophilised (freeze dried) for reconstitution prior to administration according to methods well known or apparent to those skilled in the art. Where embodiments of the invention comprise antibodies of the invention with an IgG1 isotype, a chelator of copper such as citrate (e.g. sodium citrate) or EDTA or histidine may be added to the pharmaceutical composition to reduce the degree of copper-mediated degradation of antibodies of this isotype, see EP0612251.

Effective doses and treatment regimes for administering the antibody of the invention are generally determined empirically and are dependent on factors such as the age, weight and health status of the patient and disease or disorder to be treated. Such factors are within the purview of the attending physician. Guidance in selecting appropriate doses may be found in e.g. Smith et at (1977) Antibodies in human diagnosis and therapy, Raven Press, New York but will in general be between 0.1 mg and 1 g. In one embodiment, the dosing regime for treating a human patient is 0.1 mg to 10 g of therapeutic antibody of the invention administered subcutaneously once per week or every two weeks, or by intravenous infusion every 1 or 2 months. Compositions of the present invention may also be used in prophylatically.

### 4. Clinical Uses.

The present invention relates to an antibody has the ability to bind up to four antigens selected from the group consisting of human IL-8, Gro-alpha, Gro-beta, Gro-gamma, GCP-2, NAP2, and ENA-78 The present invention also concerns methods of treating diseases or disorders characterised by elevated or unbalanced level of human IL-8, Gro-alpha, Gro-beta, Gro-gamma, GCP-2, NAP2, and/or ENA-78, particularly, COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, and/or endometriosis with said antibody, pharmaceutical compositions comprising said antibody and methods of manufacture.

The present invention also relates to use of an antibody in the manufacture of a medicament for the treatment of diseases or disorders characterised by elevated or unbalanced level of human IL-8, Gro-alpha, Gro-beta, Gro-gamma, GCP-2, NAP2, and/or ENA-78, particularly COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, or endometriosis. Although the present invention has been described principally in relation to the treatment of human diseases or disorders, the present invention may also have applications in the treatment of similar diseases or disorders in non-human mammals.

### SPECIFIC EMBODIMENTS

### Example 1

### Generation of Mouse Monoclonal Antibodies

Multiple methods and schemes were used to immunize mice in an attempt to generate mAbs of the present invention. The generation of mAbs were generated using various mixtures of multiple antigenic peptides (MAPs) and/or intact target chemokines (IL-8, Gro-α, -β, -γ, and ENA-78) mixed in complete or incomplete Freund's Adjuvant (cFA or iFA), following a modified Repetitive Immunization Multiple Sites (RIMMS) protocol in the SJL/JOrlCrl mouse strain.

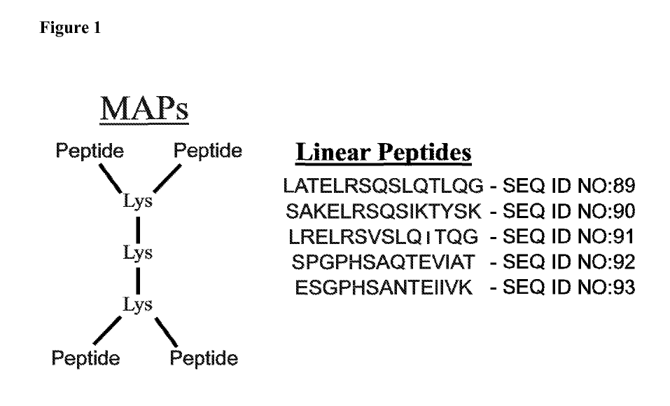

MAPs or multiple antigenic peptides serve two functions within the immunization protocol. First, MAPs allow for a selective multiple presentation of a known target amino acid sequence. Secondly, there is an increase in mass, due to multiple copies of the sequence linked, for example, via a lysine core, which also increases the immunogenicity of the sequence over that of individual peptides (Francis, J. P., et al., Immunology, 1991: 73; 249, Schott, M. E., et al., Cell. Immuno. 1996:174:199-209, Tam, J. P. Proc. Natl. Acad. Sci. 1988: 85; 5409-5413). FIG. 1 is a schematic drawing of a set of MAPs having amino acid sequences SEQ ID NOs:89-93. A linker in MAPs can be any linker other than lysines.

### General Immunization Time Line:

Two different immunization protocols following the above time line produced some mAbs of the present invention:
1. Initial immunization (day 0) consists of multiple subcutaneous injections (hind quarters, back and neck) of all target chemokines mixed in cFA (10 μg each). The following 4 boosts consisted of a mixture of all the target chemokines mixed in iFA (10 μg each). The fifth and all subsequent boosts consisted of a cocktail of all the target chemokines and all 5 linear MAPs (10 μg each) in iFA. The final boost 3 days prior to sacrifice and fusion consisted of all the target chemokines and linear MAPs in PBS and was delivered via an intraperitoneal (IP) injection.
2. Initial immunization (day 0) consists of multiple subcutaneous injections (hind quarters, back and neck) of all five linear MAPs mixed in cFA (10 μg each). The following 4 boosts consisted of a mixture of all five linear MAPs in iFA (10 μg each). The fifth and all subsequent boosts consisted of a cocktail of all the all 5 linear MAPs and all target chemokines (10 μg each) in iFA. The final boost 3 days prior to sacrifice and fusion consisted of all the all 5 linear MAPs and all target chemokines (10 μg each) in PBS and was delivered via an intraperitoneal (IP) injection.

Antibodies 1L132.23 and 1L351.17 of Table I were made by method 1 above. Antibodies 2×352.3, 2X810.3, and 2×907.15 of Table I were made by method 2 above.

### Example 2

### Functional Pan-Inhibition by Murine mAb was Confirmed Using CXCR2 Mediated Ca²⁺ Mobilization

A microtiter plate based calcium mobilization assay, FLIPR (Fluorometric Imaging Plate Reader, Molecular Devices, Sunnyvale Calif., [Schroeder, 1996]), was used for the functional characterization of the neutralizing effect of antibodies on ELR+ chemokine induced [Ca²⁺]i-mobilization in CHO-K1 cells transfected with and stably expressing hCXCR2 and Ga16.

On the day prior to assay, cells were plated in 96 well, blackwall, clear bottom plates (Packard View) at a concentration of 40000 cells per well. After 18 to 24 hours, media was aspirated off cells and replaced with 100 μl load media containing Eagles Minimal Essential Medium (EMEM) with Earl's salts and L-Glutamine, 0.1% BSA (Serologicals Corporation), 4 μM Fluo-4-acetoxymethyl ester fluorescent indicator dye (Fluo-4 AM) and 2.5 mM probenecid. Cells were incubated in this dye containing media for 1 hour at 37° C. The dye containing media was then aspirated off the cells and replaced with identical media without Fluo-4 AM and with 0.1% Gelatin (BSA removed) and 2.5 mM probenecid. Cells were incubated for 10 min. at 37° C. and then washed 3 times with KRH assay buffer [Krebs Ringer Henseleit (120 mM NaCl, 4.6 mM KCl, 1.03 mM KH₂PO₄, 25 mM NaHCO₃, 1.0 mM CaCl₂, 1.1 mM MgCl₂, 11 mM Glucose, 20 mM HEPES (pH 7.4)) with 0.1% gelatin and 2.5 mM probenecid]. After the final buffer wash, 100 μl KRH assay buffer with 0.1% gelatin and 2.5 mM probenecid was added to cells and warmed to 37° C. for 10 min. before being placed in FLIPR where dye loaded cells were exposed to excitation light (488 nm) from a 6 watt Argon Laser. [Ca²⁺]i-mobilization was monitored as a result of an increase in 516 nm emission intensity of Fluo-4 when bound to Ca²⁺. Change in emission intensity is directly related to cytosolic calcium levels, [Ca²⁺]i. After monitoring baseline for 10 sec., 50 μl of 3×ELR+ chemokine, which had been pre-incubated with a concentration range of antibody, was added to the cell plate and data collected every sec. for 1 min., followed by an additional half min. of recording in 3 sec. intervals. Maximal cellular response above basal reading was exported for plotting in GraphPad Prism (v4.03).

The IC₅₀ was defined as the concentration of antibody required, during pre-treatment of 3×EC₈₀ chemokine, to neutralize the CXCR2 mediated stimulatory effect of an EC₈₀ concentration of the ELR+ chemokine by 50%. A secondary cellular response to 25 μM ATP was monitored to test cell viability [Sarau, 1999].

Schroeder K S, Neagle, B D. FLIPR: a new instrument for accurate, high throughput optical screening. J. Biomol. Screen. 1996:1-75.
Sarau H M, Ames R S, Chambers J, Ellis C, Elshourbagy N, Foley J J et al. Identification, molecular cloning, expression, and characterization of a cysteinyl leukotrien receptor. Mol. Pharmacol. 1999; 56:657-663.

TABLE I
FLIPR IC50 (ug/ml) on purified Mabs**

| | IL8 | GroA | GroB | GroG | ENA78 | GCP2 | NAP2 | isotype |
|---|---|---|---|---|---|---|---|---|
| 5D2.2D10 | >200 | >200 | 77.4 | >200 | 4.7 | >200 | >200 | IgG1 |
| 1A1.4C6 | >200 | 2.4 | >167.5 | 19.2 | 12.3 | 11.2 | 122.9 | IgG1 |
| 7A12.1F10 | >200 | 9.9 | >200 | >200 | 2.4 | 123.6 | >200 | IgG1 |
| 9D12.1H4 | 3.4 | >67 | >67 | >67 | 14.4 | 6.6 | >67 | IgG1 |
| 2B12.1E1 | 1.6 | >67 | >65.5 | >67 | >67 | 40.9 | >67 | IgG2c |
| 17D1.1D2 | 2.5 | >146.3 | 81.6 | 143.9 | >200 | >200 | >200 | IgG2c |
| 1L132.23 | >67 | >67 | 0.69 | >67 | >67 | >67 | >67 | IgG1 |
| 1L351.17 | >67 | 1.92 | 1.02 | 3.17 | >67 | >67 | >67 | IgG2b |
| 2X352.3 | >67 | 2.06 | 1.12 | 7.43 | 4.99 | >67 | >67 | IgG1 |
| 2X810.3 | >67 | >67 | >67 | >67 | 14.57 | 24.59 | >67 | IgG1 |
| 2X907.15 | >67 | >67 | >67 | >67 | 2.6 | >67* | >67 | IgG1 |

**In this assay values over 122 ug/ml are considered not to be active.
*partial

Complementarity Determining Regions (CDRs) are underlined.

5D2.2D10 heavy chain
Sequence Listing No. 1
QIQLVQSGPELKKPGETVKISCKASGYIFTAYGMSWVKQAPGKGLKWMG
WINTYSGVPTYADHFKGRFDFSLETSASTAYLQINNLKNEDTATYFCAR
PTVVDPWYFDVWGTGTTVTVSS
5D2.2D10 light chain
Sequence Listing No. 2
DIQMTQSPASLSVSVGETVTITCRASENIYSNLAWYQQKQGKSPQLLVY
AATNLGDGVPSRFSGSGSGTQYSLKINSLQSEDFGSYYCQHFWTTPWTF
GGGTKLEIK
1A1.4C6 heavy chain
Sequence Listing No. 3
EVQLQQSGPELVKPGASVKISCKASGYTFIDYYMNWVKQSQGKSLEWIG
DVNPDDGDTTYNQQFKDKATLTVDKSSSTAYMELRSLTSEDSAVYYCAR
DDRTSGYFDVWGTGTTVTVSS
1A1.4C6 light chain
Sequence Listing No. 4
DIQMTQSTSSLSASLGDRVTISCRASQDIRNYLNWFQQKPDGTVKLLIY
YTSIVHSRVPSRFSGSGSGTDYSLTINNLDQEDIATYFCQQANTLPWTF
GGGTKLETK
7A12.1F10 heavy chain
Sequence Listing No. 5
QVQLQQSGAELVKPGASVKLSCKASGYIFTSNDINWVRQRPEQGLEWIG
WIFPGDNSTKFNEKFRDKATLTTDKSSSTAYMQLSGLTSEDSAVYFCAT
FYGSTYGYYFDYWGQGTTLTVSS
7Al2.1F10 light chain
Sequence Listing No. 6
DIVMTQSHKFMSTSVGDRVSITCKASQDVGTAVAWYQQKPGQSPKLLIY
WTDTRHTGVPDRFTGSGSGTDFTLTISNVESEDCVDYFCHQYNNYPLTF
GAGTKLELK
9D12.1H4 heavy chain
Sequence Listing No. 7
EVQLQQSGPELVKPGASVRMSCKASGNTVIDYNIHWVKQSQGKSLEWIG
YINPNSGGTGYNEKFKDKATLTINKSSSTAHMDLRSLTSEDSAVYYCTR
FDFWGQGTTLTVSS
9D12.1H4 light chain
Sequence Listing No. 8
DVLMTQTPLSLPVSLGDQASISCRSTQSIVPVNGNTHLEWYLHKPGQSP
KLLIYKVSNRFSGVPDRFSGSGSGTDFTLKISRVEAEDLGVYYCFQASH
VPWTFGGGTKLEIK
2B12.1E1 heavy chain
Sequence Listing No. 9
EVQLQQSGPELVKPGASVKISCKASGYTFTDYYMNWVKQSHGKSLEWIG
DINPNNGNTNYNQKFKGKATLTVDKSSSTAYMEFRSLTSEDSAVYYCAG
LGRIFDYWGQGTTLTVSS
2B12.1E1 light chain
Sequence Listing No. 10
ENVLTQSPAIMSASPGEKVTMTCRASSSVSSTFLHWYQQKSGASPKLWI
YSTSDLASGVPARFSGSGSGTSYSLTISSVEAEDAATYYCQQYSGYPWT
FGGGTKLEIK
17D1.1D2 heavy chain
Sequence Listing No. 11
EVQLQQSGPELVKPGASVKISCKASGYTFTDYYMNWLRQSHGKSLEWIG
DINPNNGGTNYNQKFKNKATLTVDKSSSTAYMELRSLTSEDSAVFYCAG
LGRIFDYWGQGTTLTVSS
17D1.1D2 light chain
Sequence Listing No. 12
ENVLTQSPAIMSAFPGEKVTMTCRASSSVSSTYLHWYQQKSGASPKLWI
YSTSDLASGVPARFSGSGSGTSYSLTISSVEAEDAATYYCQQFSGYPWT
FGGGTKLEIK
1L132.23 heavy chain
Sequence Listing No. 13
QVQLQQPGAELVKPGASVKVSCKASGYTFTNYWMHWVRQRPGQGLEWIG
RIHPSDNDTNYNQKFKDKATLTVDKSSNTAYMQLSSLTSEDSAVYYCAI
GVYDGFIGLWGQGTSVTVSS
1L132.23 light chain
Sequence Listing No. 14
DIQMTQSPASQSASLGESVTITCLASQTIGTWLAWYQQKPGKSPQLLIY
AATRLADGVPSRFSGSGSGTKFSFKISSLQAEDFVSYYCQQLSSTPWTF
GGGTKLEIK
1L351.17 heavy chain
Sequence Listing No. 15
QAYLQQSGAELVRPGASVKMSCKASGYTFTSYNMHWVKQTPRQGLEWIG
AIYPGNGDTSYNQKFKGKATLTVDKSSSTAYMQLSSLTSEDSAVYFCAR
DLYYFDYWGQGTTLTVSS
1L351.17 light chain
Sequence Listing No. 16
ENVLTQSPAIMSAFPGEKVTMTCRASSSVSSTYLHWYQQKSGASPKLWI
YSTSDLASGVPARFSGSGSGTSYSLTISSVEAEDAATYYCQQFSGYPWT
FGGGTKLEIK
2X352.3 heavy chain
Sequence Listing No. 17
QIQLVQSGPELKKPGETVKISCKASGYTLTYYGIHWVTQTPGKGLNWMG
WINTNTGEPTYVEEFKGRFAFSLETSASTAYLQITDLKNEDTATYFCAK
ATYDGYSDYWGQGTTLTVSS
2X352.3 light chain
Sequence Listing No. 18
DIQMTQSPASLSVSVGESVTITCRASENIYSNLAWYQQKQGKSPQLLVY
DATNLAHGVPSRFSGSGSGTQFSLRINSLQSEDFGSYYCQHFWGTPLTF
GAGTRLELK
2X810.3 heavy chain
Sequence Listing No. 19
QIQLQQSGPEVVKPGASVKISCKASGYIFTDNYIDWVQQRPGQGLEWIG
WIFPGSGNTKYNEKFKGKATLTVDTFSSTAYMQLSSLTSEDTAVYFCAR
EIDYDYDGFFDVWGAGTTVTVSS
2X810.3 light chain
Sequence Listing No. 20
DIVMTPSHTFMSTSVGDRVIITSKASQDVGSAVAWYQQKPGQSPTLLIY
WASTRHTGVPDRFTGSGSGTDFTLTISNLQSEDLADYFCQQYSTYPLTF
GAGTKLELK
2X907.15 heavy chain
Sequence Listing No. 21
EVKLVESGGGLVKPGGSLKLSCAASGFTFSDYYMYWVRQSPEKRLEWVA
TVSDVGSYTYYSGSVRGRFTISRDNAKNNLYLQMSSLQSEDTAIYYCSR
DRTLDYWGQGTSVIVTS
2X907.15 light chain
Sequence Listing No. 22
QIVLTQSPAIMSASPGEKVTITCNASSSVSYMHWFQQKPGTSPKLWIYS
TSNLASGVPARFSGSGSGTSYSLTISRMEAEDAATYYCHQRSSFPPTFG
GGTKLEIK
Sequence Listing No. 23
AYGMS
Sequence Listing No. 24
WINTYSGVPTYADHFKG
Sequence Listing No. 25
PTVVDPWYFDV
Sequence Listing No. 26
RASENIYSNLA
Sequence Listing No. 27
AATNLGD
Sequence Listing No. 28
QHFWTTPWT
Sequence Listing No. 29
DYYMN
Sequence Listing No. 30
DVNPDDGDTTYNQQFKD
Sequence Listing No. 31
DDRTSGYFDV
Sequence Listing No. 32
RASQDIRNYLN
Sequence Listing No. 33
YTSIVHS
Sequence Listing No. 34
QQANTLPWT
Sequence Listing No. 35
SNDIN
Sequence Listing No. 36
WIFPGDNSTKFNEKFRD
Sequence Listing No. 37
FYGSTYGYYFDY
Sequence Listing No. 38
KASQDVGTAVA
Sequence Listing No. 39
WTDTRHT
Sequence Listing No. 40
HQYNNYPLT
Sequence Listing No. 41
DYNIH
Sequence Listing No. 42
YINPNSGGTGYNEKFKD
Sequence Listing No. 43
FDF
Sequence Listing No. 44
RSTQSIVPVNGNTHLE
Sequence Listing No. 45
KVSNRFS
Sequence Listing No. 46
FQASHVPWT
Sequence Listing No. 47
DYYMN
Sequence Listing No. 48
DINPNNGNTNYNQKFKG
Sequence Listing No. 49
LGRIFDY
Sequence Listing No. 50
RASSSVSSTFLH
Sequence Listing No. 51
STSDLAS
Sequence Listing No. 52
QQYSGYPWT
Sequence Listing No. 53
DYYMN
Sequence Listing No. 54
DINPNNGGTNYNQKFKN
Sequence Listing No. 55
LGRIFDY
Sequence Listing No. 56
RASSSVSSTYLH
Sequence Listing No. 57
STSDLAS
Sequence Listing No. 58
QQFSGYPWT
Sequence Listing No. 59
NYWMH
Sequence Listing No. 60
RIHPSDNDTNYNQKFKD
Sequence Listing No. 61
GVYDGFIGL
Sequence Listing No. 62
LASQTIGTWLA
Sequence Listing No. 63
AATRLAD
Sequence Listing No. 64
QQLSSTPWT
Sequence Listing No. 65
SYNMH
Sequence Listing No. 66
AIYPGNGDTSYNQKFKG
Sequence Listing No. 67
DLYYFDY
Sequence Listing No. 68
RASSSVSSTYLH
Sequence Listing No. 69
STSDLAS
Sequence Listing No. 70
QQFSGYPWT
Sequence Listing No. 71
YYGIH
Sequence Listing No. 72
WINTNTGEPTYVEEFKG
Sequence Listing No. 73
ATYDGYSDY
Sequence Listing No. 74
RASENIYSNLA
Sequence Listing No. 75
DATNLAH
Sequence Listing No. 76
QHFWGTPLT
Sequence Listing No. 77
DNYID
Sequence Listing No. 78
WIFPGSGNTKYNEKFKG
Sequence Listing No. 79
EIDYDYDGFFDV
Sequence Listing No. 80
KASQDVGSAVA
Sequence Listing No. 81
WASTRHT
Sequence Listing No. 82
QQYSTYPLT
Sequence Listing No. 83
DYYMY
Sequence Listing No. 84
TVSDVGSYTYYSGSVRG
Sequence Listing No. 85
DRTLDY
Sequence Listing No. 86
NASSSVSYMH
Sequence Listing No. 87
STSNLAS
Sequence Listing No. 88
HQRSSFPPT

1. An antibody comprising heavy and light chain variable regions comprising
(a) the CDR amino acid sequences of SEQ ID NOs: 23, 24, and 25, and SEQ ID NOs: 26, 27, and 28, respectively, or one or more of the CDR sequences can be conservative sequence modifications of the sequences listed in SEQ ID NOs: 23, 24, 25, 26, 27, and 28; or
(b) the CDR amino acid sequences of SEQ ID NOs: 29, 30, and 31, and SEQ ID NOs: 32, 33, and 34, respectively; or one or more of the CDR sequences can be conservative sequence modifications of the sequences listed in SEQ ID NOs: 29, 30, 31, 32, 33, and 34; or
(c) the CDR amino acid sequences of SEQ ID NOs: 35, 36, and 37, and SEQ ID NOs: 38, 39, and 40, respectively; or one or more of the CDR sequences can be conservative sequence modifications of the sequences listed in SEQ ID NOs: 35, 36, 37, 38, 39, and 40; or
(d) the CDR amino acid sequences of SEQ ID NOs: 41, 42, and 43, and SEQ ID NOs: 44, 45, and 46, respectively; or one or more of the CDR sequences can be conservative sequence modifications of the sequences listed in SEQ ID NOs: 41, 42, 43, 44, 45, and 46; or
(e) the CDR amino acid sequences of SEQ ID NOs: 47, 48, and 49, and SEQ ID NOs: 50, 51, and 52, respectively; or one or more of the CDR sequences can be conservative sequence modifications of the sequences listed in SEQ ID NOs: 47, 48, 49, 50, 51, and 52; or
(f) the CDR amino acid sequences of SEQ ID NOs: 53, 54, and 55, and SEQ ID NOs: 56, 57, and 58, respectively; or one or more of the CDR sequences can be conservative sequence modifications of the sequences listed in SEQ ID NOs: 53, 54, 55, 56, 57, and 58; or
(g) the CDR amino acid sequences of SEQ ID NOs: 59, 60, and 61, and SEQ ID NOs: 62, 63, and 64, respectively; or one or more of the CDR sequences can be conservative sequence modifications of the sequences listed in SEQ ID NOs: 59, 60, 61, 62, 63, and 64; or
(h) the CDR amino acid sequences of SEQ ID NOs: 65, 66, and 67, and SEQ ID NOs: 68, 69, and 70, respectively; or one or more of the CDR sequences can be conservative sequence modifications of the sequences listed in SEQ ID NOs: 65, 66, 67, 68, 69, and 70; or
(i) the CDR amino acid sequences of SEQ ID NOs: 71, 72, and 73, and SEQ ID NOs: 74, 75, and 76, respectively; or one or more of the CDR sequences can be conservative sequence modifications of the sequences listed in SEQ ID NOs: 71, 72, 73, 74, 75, and 76; or
(j) the CDR amino acid sequences of SEQ ID NOs: 77, 78, and 79, and SEQ ID NOs: 80, 81, and 82, respectively; or one or more of the CDR sequences can be conservative sequence modifications of the sequences listed in SEQ ID NOs: 77, 78, 79, 80, 81, and 82; or
(k) the CDR amino acid sequences of SEQ ID NOs: 83, 84, and 85, and SEQ ID NOs: 86, 87, and 88, respectively; or one or more of the CDR sequences can be conservative sequence modifications of the sequences listed in SEQ ID NOs: 83, 84, 85, 86, 87, and 88.

2. An expression vector comprising nucleotide sequence encoding a variable heavy or light chain of claim 1.

3. A recombinant host cell which produces an antibody of claim 1.

4. A method of treating or preventing COPD, osteoarthritis, rheumatoid arthritis, erosive arthritis, asthma, atherosclerosis, inflammatory bowel disease (including ulcerative colitis), psoriasis, transplant rejection, gout, cancer, acute lung injury, acute lung disease, sepsis, ARDS, peripheral artery disease, systemic sclerosis, neonatal respiratory distress syndrome, exacerbation of asthma and COPD, cystic fibrosis, diffuse panbronchiolitis, reperfusion injury, or endometriosis in a human patient with a therapeutic effective amount of antibody of claim 1.


SEQUENCE LISTING (93 sequences)

SEQ ID NO: 1 - protein, 120 aa, Artificial Sequence
  Murine
QIQLVQSGPELKKPGETVKISCKASGYIFTAYGMSWVKQAPGKGLKWMGWINTYSGVPTYADHFKGRFDFSLETSASTAYLQINNLKNEDTATYFCARPTVVDPWYFDVWGTGTTVTVSS

SEQ ID NO: 2 - protein, 107 aa, Artificial Sequence
  Murine
DIQMTQSPASLSVSVGETVTITCRASENIYSNLAWYQQKQGKSPQLLVYAATNLGDGVPSRFSGSGSGTQYSLKINSLQSEDFGSYYCQHFWTTPWTFGGGTKLEIK

SEQ ID NO: 3 - protein, 119 aa, Artificial Sequence
  Murine
EVQLQQSGPELVKPGASVKISCKASGYTFIDYYMNWVKQSQGKSLEWIGDVNPDDGDTTYNQQFKDKATLTVDKSSSTAYMELRSLTSEDSAVYYCARDDRTSGYFDVWGTGTTVTVSS

SEQ ID NO: 4 - protein, 107 aa, Artificial Sequence
  Murine
DIQMTQSTSSLSASLGDRVTISCRASQDIRNYLNWFQQKPDGTVKLLIYYTSIVHSRVPSRFSGSGSGTDYSLTINNLDQEDIATYFCQQANTLPWTFGGGTKLETK

SEQ ID NO: 5 - protein, 121 aa, Artificial Sequence
  Murine
QVQLQQSGAELVKPGASVKLSCKASGYIFTSNDINWVRQRPEQGLEWIGWIFPGDNSTKFNEKFRDKATLTTDKSSSTAYMQLSGLTSEDSAVYFCATFYGSTYGYYFDYWGQGTTLTVSS

SEQ ID NO: 6 - protein, 107 aa, Artificial Sequence
  Murine
DIVMTQSHKFMSTSVGDRVSITCKASQDVGTAVAWYQQKPGQSPKLLIYWTDTRHTGVPDRFTGSGSGTDFTLTISNVESEDCVDYFCHQYNNYPLTFGAGTKLELK

SEQ ID NO: 7 - protein, 112 aa, Artificial Sequence
  Murine
EVQLQQSGPELVKPGASVRMSCKASGNTVIDYNIHWVKQSQGKSLEWIGYINPNSGGTGYNEKFKDKATLTINKSSSTAHMDLRSLTSEDSAVYYCTRFDFWGQGTTLTVSS

SEQ ID NO: 8 - protein, 112 aa, Artificial Sequence
  Murine
DVLMTQTPLSLPVSLGDQASISCRSTQSIVPVNGNTHLEWYLHKPGQSPKLLIYKVSNRFSGVPDRFSGSGSGTDFTLKISRVEAEDLGVYYCFQASHVPWTFGGGTKLEIK

SEQ ID NO: 9 - protein, 116 aa, Artificial Sequence
  Murine
EVQLQQSGPELVKPGASVKISCKASGYTFTDYYMNWVKQSHGKSLEWIGDINPNNGNTNYNQKFKGKATLTVDKSSSTAYMEFRSLTSEDSAVYYCAGLGRIFDYWGQGTTLTVSS

SEQ ID NO: 10 - protein, 108 aa, Artificial Sequence
  Murine
ENVLTQSPAIMSASPGEKVTMTCRASSSVSSTFLHWYQQKSGASPKLWIYSTSDLASGVPARFSGSGSGTSYSLTISSVEAEDAATYYCQQYSGYPWTFGGGTKLEIK

SEQ ID NO: 11 - protein, 116 aa, Artificial Sequence
  Murine
EVQLQQSGPELVKPGASVKISCKASGYTFTDYYMNWLRQSHGKSLEWIGDINPNNGGTNYNQKFKNKATLTVDKSSSTAYMELRSLTSEDSAVFYCAGLGRIFDYWGQGTTLTVSS

SEQ ID NO: 12 - protein, 108 aa, Artificial Sequence
  Murine
ENVLTQSPAIMSAFPGEKVTMTCRASSSVSSTYLHWYQQKSGASPKLWIYSTSDLASGVPARFSGSGSGTSYSLTISSVEAEDAATYYCQQFSGYPWTFGGGTKLEIK

SEQ ID NO: 13 - protein, 118 aa, Artificial Sequence
  Murine
QVQLQQPGAELVKPGASVKVSCKASGYTFTNYWMHWVRQRPGQGLEWIGRIHPSDNDTNYNQKFKDKATLTVDKSSNTAYMQLSSLTSEDSAVYYCAIGVYDGFIGLWGQGTSVTVSS

SEQ ID NO: 14 - protein, 107 aa, Artificial Sequence
  Murine
DIQMTQSPASQSASLGESVTITCLASQTIGTWLAWYQQKPGKSPQLLIYAATRLADGVPSRFSGSGSGTKFSFKISSLQAEDFVSYYCQQLSSTPWTFGGGTKLEIK

SEQ ID NO: 15 - protein, 116 aa, Artificial Sequence
  Murine
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARDLYYFDYWGQGTTLTVSS

SEQ ID NO: 16 - protein, 108 aa, Artificial Sequence; identical to SEQ ID NO: 12
  Murine

SEQ ID NO: 17 - protein, 118 aa, Artificial Sequence
  Murine
QIQLVQSGPELKKPGETVKISCKASGYTLTYYGIHWVTQTPGKGLNWMGWINTNTGEPTYVEEFKGRFAFSLETSASTAYLQITDLKNEDTATYFCAKATYDGYSDYWGQGTTLTVSS

SEQ ID NO: 18 - protein, 107 aa, Artificial Sequence
  Murine
DIQMTQSPASLSVSVGESVTITCRASENIYSNLAWYQQKQGKSPQLLVYDATNLAHGVPSRFSGSGSGTQFSLRINSLQSEDFGSYYCQHFWGTPLTFGAGTRLELK

SEQ ID NO: 19 - protein, 121 aa, Artificial Sequence
  Murine
QIQLQQSGPEVVKPGASVKISCKASGYIFTDNYIDWVQQRPGQGLEWIGWIFPGSGNTKYNEKFKGKATLTVDTFSSTAYMQLSSLTSEDTAVYFCAREIDYDYDGFFDVWGAGTTVTVSS

SEQ ID NO: 20 - protein, 107 aa, Artificial Sequence
  Murine
DIVMTPSHTFMSTSVGDRVIITSKASQDVGSAVAWYQQKPGQSPTLLIYWASTRHTGVPDRFTGSGSGTDFTLTISNLQSEDLADYFCQQYSTYPLTFGAGTKLELK

SEQ ID NO: 21 - protein, 115 aa, Artificial Sequence
  Murine
EVKLVESGGGLVKPGGSLKLSCAASGFTFSDYYMYWVRQSPEKRLEWVATVSDVGSYTYYSGSVRGRFTISRDNAKNNLYLQMSSLQSEDTAIYYCSRDRTLDYWGQGTSVIVTS

SEQ ID NO: 22 - protein, 106 aa, Artificial Sequence
  Murine
QIVLTQSPAIMSASPGEKVTITCNASSSVSYMHWFQQKPGTSPKLWIYSTSNLASGVPARFSGSGSGTSYSLTISRMEAEDAATYYCHQRSSFPPTFGGGTKLEIK

SEQ ID NO: 23 - protein, 5 aa, Artificial Sequence
  CDRH1
AYGMS

SEQ ID NO: 24 - protein, 17 aa, Artificial Sequence
  CDRH2
WINTYSGVPTYADHFKG

SEQ ID NO: 25 - protein, 11 aa, Artificial Sequence
  CDRH3
PTVVDPWYFDV

SEQ ID NO: 26 - protein, 11 aa, Artificial Sequence
  CDRL1
RASENIYSNLA

SEQ ID NO: 27 - protein, 7 aa, Artificial Sequence
  CDRL2
AATNLGD

SEQ ID NO: 28 - protein, 9 aa, Artificial Sequence
  CDRL3
QHFWTTPWT

SEQ ID NO: 29 - protein, 5 aa, Artificial Sequence
  CDRH1
DYYMN

SEQ ID NO: 30 - protein, 17 aa, Artificial Sequence
  CDRH2
DVNPDDGDTTYNQQFKD

SEQ ID NO: 31 - protein, 10 aa, Artificial Sequence
  CDRH3
DDRTSGYFDV

SEQ ID NO: 32 - protein, 11 aa, Artificial Sequence
  CDRL1
RASQDIRNYLN

SEQ ID NO: 33 - protein, 7 aa, Artificial Sequence
  CDRL2
YTSIVHS

SEQ ID NO: 34 - protein, 9 aa, Artificial Sequence
  CDRL3
QQANTLPWT

SEQ ID NO: 35 - protein, 5 aa, Artificial Sequence
  CDRH1
SNDIN

SEQ ID NO: 36 - protein, 17 aa, Artificial Sequence
  CDRH2
WIFPGDNSTKFNEKFRD

SEQ ID NO: 37 - protein, 12 aa, Artificial Sequence
  CDRH3
FYGSTYGYYFDY

SEQ ID NO: 38 - protein, 11 aa, Artificial Sequence
  CDRL1
KASQDVGTAVA

SEQ ID NO: 39 - protein, 7 aa, Artificial Sequence
  CDRL2
WTDTRHT

SEQ ID NO: 40 - protein, 9 aa, Artificial Sequence
  CDRL3
HQYNNYPLT

SEQ ID NO: 41 - protein, 5 aa, Artificial Sequence
  CDRH1
DYNIH

SEQ ID NO: 42 - protein, 17 aa, Artificial Sequence
  CDRH2
YINPNSGGTGYNEKFKD

SEQ ID NO: 43 - protein, 3 aa, Artificial Sequence
  CDRH3
FDF

SEQ ID NO: 44 - protein, 16 aa, Artificial Sequence
  CDRL1
RSTQSIVPVNGNTHLE

SEQ ID NO: 45 - protein, 7 aa, Artificial Sequence
  CDRL2
KVSNRFS

SEQ ID NO: 46 - protein, 9 aa, Artificial Sequence
  CDRL3
FQASHVPWT

SEQ ID NO: 47 - protein, 5 aa, Artificial Sequence
  CDRH1
DYYMN

SEQ ID NO: 48 - protein, 17 aa, Artificial Sequence
  CDRH2
DINPNNGNTNYNQKFKG

SEQ ID NO: 49 - protein, 7 aa, Artificial Sequence
  CDRH3
LGRIFDY

SEQ ID NO: 50 - protein, 12 aa, Artificial Sequence
  CDRL1
RASSSVSSTFLH

SEQ ID NO: 51 - protein, 7 aa, Artificial Sequence
  CDRL2
STSDLAS

SEQ ID NO: 52 - protein, 9 aa, Artificial Sequence
  CDRL3
QQYSGYPWT

SEQ ID NO: 53 - protein, 5 aa, Artificial Sequence
  CDRH1
DYYMN

SEQ ID NO: 54 - protein, 17 aa, Artificial Sequence
  CDRH2
DINPNNGGTNYNQKFKN

SEQ ID NO: 55 - protein, 7 aa, Artificial Sequence
  CDRH3
LGRIFDY

SEQ ID NO: 56 - protein, 12 aa, Artificial Sequence
  CDRL1
RASSSVSSTYLH

SEQ ID NO: 57 - protein, 7 aa, Artificial Sequence
  CDRL2
STSDLAS

SEQ ID NO: 58 - protein, 9 aa, Artificial Sequence
  CDRL3
QQFSGYPWT

SEQ ID NO: 59 - protein, 5 aa, Artificial Sequence
  CDRH1
NYWMH

SEQ ID NO: 60 - protein, 17 aa, Artificial Sequence
  CDRH2
RIHPSDNDTNYNQKFKD

SEQ ID NO: 61 - protein, 9 aa, Artificial Sequence
  CDRH3
GVYDGFIGL

SEQ ID NO: 62 - protein, 11 aa, Artificial Sequence
  CDRL1
LASQTIGTWLA

SEQ ID NO: 63 - protein, 7 aa, Artificial Sequence
  CDRL2
AATRLAD

SEQ ID NO: 64 - protein, 9 aa, Artificial Sequence
  CDRL3
QQLSSTPWT

SEQ ID NO: 65 - protein, 5 aa, Artificial Sequence
  CDRH1
SYNMH

SEQ ID NO: 66 - protein, 17 aa, Artificial Sequence
  CDRH2
AIYPGNGDTSYNQKFKG

SEQ ID NO: 67 - protein, 7 aa, Artificial Sequence
  CDRH3
DLYYFDY

SEQ ID NO: 68 - protein, 12 aa, Artificial Sequence
  CDRL1
RASSSVSSTYLH

SEQ ID NO: 69 - protein, 7 aa, Artificial Sequence
  CDRL2
STSDLAS

SEQ ID NO: 70 - protein, 9 aa, Artificial Sequence
  CDRL3
QQFSGYPWT

SEQ ID NO: 71 - protein, 5 aa, Artificial Sequence
  CDRH1
YYGIH

SEQ ID NO: 72 - protein, 17 aa, Artificial Sequence
  CDRH2
WINTNTGEPTYVEEFKG

SEQ ID NO: 73 - protein, 9 aa, Artificial Sequence
  CDRH3
ATYDGYSDY

SEQ ID NO: 74 - protein, 11 aa, Artificial Sequence
  CDRL1
RASENIYSNLA

SEQ ID NO: 75 - protein, 7 aa, Artificial Sequence
  CDRL2
DATNLAH

SEQ ID NO: 76 - protein, 9 aa, Artificial Sequence
  CDRL3
QHFWGTPLT

SEQ ID NO: 77 - protein, 5 aa, Artificial Sequence
  CDRH1
DNYID

SEQ ID NO: 78 - protein, 17 aa, Artificial Sequence
  CDRH2
WIFPGSGNTKYNEKFKG

SEQ ID NO: 79 - protein, 12 aa, Artificial Sequence
  CDRH3
EIDYDYDGFFDV

SEQ ID NO: 80 - protein, 11 aa, Artificial Sequence
  CDRL1
KASQDVGSAVA

SEQ ID NO: 81 - protein, 7 aa, Artificial Sequence
  CDRL2
WASTRHT

SEQ ID NO: 82 - protein, 9 aa, Artificial Sequence
  CDRL3
QQYSTYPLT

SEQ ID NO: 83 - protein, 5 aa, Artificial Sequence
  CDRH1
DYYMY

SEQ ID NO: 84 - protein, 17 aa, Artificial Sequence
  CDRH2
TVSDVGSYTYYSGSVRG

SEQ ID NO: 85 - protein, 6 aa, Artificial Sequence
  CDRH3
DRTLDY

SEQ ID NO: 86 - protein, 10 aa, Artificial Sequence
  CDRL1
NASSSVSYMH

SEQ ID NO: 87 - protein, 7 aa, Artificial Sequence
  CDRL2
STSNLAS

SEQ ID NO: 88 - protein, 9 aa, Artificial Sequence
  CDRL3
HQRSSFPPT

SEQ ID NO: 89 - protein, 15 aa, Artificial Sequence
  Linear peptide
LATELRSQSLQTLQG

SEQ ID NO: 90 - protein, 15 aa, Artificial Sequence
  Linear peptide
SAKELRSQSIKTYSK

SEQ ID NO: 91 - protein, 14 aa, Artificial Sequence
  Linear peptide
LRELRSVSLQITQG

SEQ ID NO: 92 - protein, 14 aa, Artificial Sequence
  Linear peptide
SPGPHSAQTEVIAT

SEQ ID NO: 93 - protein, 14 aa, Artificial Sequence
  Linear peptide
ESGPHSANTEIIVK